SEQUENCE LISTING (62 sequences)

SEQ ID NO: 1 - protein, 113 aa, synthetic construct
QVQLQQSGAELVRPGASVTLSCKASDYTFTDYEMHWVKQTPVHGLEWIGAIDPEAGGTDYNQKFKGKAILTADKSSSTAYMELRSLTSEDSAVYYCTRVWLHWGQGTTLTVSS

SEQ ID NO: 2 - protein, 112 aa, synthetic construct
NIVMTQSPSSLAVSAGEKVTMNCKSSQSVLYSSNQKNYLAWYQQKPGQSPKMLIYWASTRESGVPDRFTGSGSGTDFTLTISSVQSEDLAVYYCHQYLSSNTFGGGTKLEIK

SEQ ID NO: 3 - protein, 5 aa, synthetic construct
DYEMH

SEQ ID NO: 4 - protein, 17 aa, synthetic construct
AIDPEAGGTDYNQKFKG

SEQ ID NO: 5 - protein, 4 aa, synthetic construct
VWLH

SEQ ID NO: 6 - protein, 118 aa, synthetic construct
QVQLVQSGAEVKKPGSSVKVSCKASGYDFSNYWMNWVRQAPGQGLEWMGQIYPGDGEIKYNQKFQGRVTITADESTSTAYMELSSLRSEDTAVYYCARDDYLRAMDYWGQGTLVTVSS

SEQ ID NO: 7 - protein, 118 aa, synthetic construct
QVQLVQSGAEVKKPGSSVKVSCKASGYDFSNFWMNWVRQAPGQGLEWMGQIYPGDGEIKYNQKFQGRVTITADESTSTAYMELSSLRSEDTAVYYCARDDYLRAMDYWGQGTLVTVSS

SEQ ID NO: 8 - protein, 118 aa, synthetic construct
QVQLVQSGAEVKKPGSSVKVSCKASGYDFSNFWMNWVRQAPGQGLEWMGQIYPGDGEIKYAQKFQGRVTITADESTSTAYMELSSLRSEDTAVYYCARDDYLRAMDIWGQGTMVTVSS

SEQ ID NO: 9 - protein, 107 aa, synthetic construct
DIQMTQSPSSLSASVGDRVTITCRASQNIHNYLAWYQQKPGKAPKFLLYSAKRLESGVPSRFSGSGSGTDYTLTISSLQPEDFATYYCQHFWSSPYTFGQGTKLEIK

SEQ ID NO: 10 - protein, 107 aa, synthetic construct
DIQMTQSPSSLSASVGDRVTITCRASGNIHNYLAWYQQKPGKAPKFLLYSAKRLESGVPSRFSGSGSGTDYTLTISSLQPEDFATYYCQHFWSSPYTFGGGTKVEIK

SEQ ID NO: 11 - protein, 8 aa, synthetic construct
DYTFTDYE

SEQ ID NO: 12 - protein, 8 aa, synthetic construct
IDPEAGGT

SEQ ID NO: 13 - protein, 6 aa, synthetic construct
TRVWLH

SEQ ID NO: 14 - protein, 12 aa, synthetic construct
QSVLYSSNQKNY

SEQ ID NO: 15 - protein, 8 aa, synthetic construct
DYTFTGYE

SEQ ID NO: 16 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 17 - protein, 113 aa, synthetic construct
QVQLVQSGAEVKKPGASVKVSCKASDYTFTGYEMHWVRQAPGQGLEWMGAIDPEAGGTDYAQKFQGRVTMTADTSISTAYMELSRLRSDDTAVYYCTRVWLHWGQGTLVTVSS

SEQ ID NO: 18 - protein, 112 aa, synthetic construct
DIVMTQSPDSLAVSLGERVTMNCKSSQSVLYSSNQKNYLAWYQQKPGQPPKMLIYWASTRESGVPDRFSGSGSGTDFTLTISSLQAEDVAVYYCHQYLSSNTFGQGTKLEIK

SEQ ID NO: 19 - protein, 113 aa, synthetic construct
QVQLVQSGAEVKKPGASVKVSCKASDYTFTDYEMHWVRQAPGQGLEWMGAIDPEAGGTDYAQKFQGRVTMTADTSISTAYMELSRLRSDDTAVYYCTRVWLHWGQGTLVTVSS

SEQ ID NO: 20 - protein, 467 aa, Homo sapiens
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 21 - protein, 8 aa, synthetic construct
  VARIANT 6: note = Glycine or Aspartic acid
DYTFTXYE

SEQ ID NO: 22 - protein, 118 aa, synthetic construct
QVQLHQSGAELVKPGASVKISCKGSGYDFSNFWMNWVKQRPGKGLEWIGQIYPGDGEIKYNGKFKGKATLTADESSSTAYIHLSSLTSEDSAVYFCARDDYLRAMDYWGQGTSVTVSS

SEQ ID NO: 23 - protein, 107 aa, synthetic construct
DIQMTQSPASLSASVGETVTITCRASGNIHNYLAWFQQKQGKSPHFLVYSAKALADGVPSRFSGSGSGTQYSLKINSLQPEDFGTYYCQHFWSSPYTFGGGTKLEIK

SEQ ID NO: 24 - protein, 110 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 25 - protein, 17 aa, synthetic construct
KSSQSVLYSSNQKNYLA

SEQ ID NO: 26 - protein, 7 aa, synthetic construct
WASTRES

SEQ ID NO: 27 - protein, 8 aa, synthetic construct
HQYLSSNT

SEQ ID NO: 28 - protein, 7 aa, synthetic construct
DYTFTDY

SEQ ID NO: 29 - protein, 6 aa, synthetic construct
DPEAGG

SEQ ID NO: 30 - protein, 9 aa, synthetic construct
  VARIANT 9: note = Tyrosine, Valine or Isoleucine
DDYLRAMDX

SEQ ID NO: 31 - protein, 7 aa, synthetic construct
GYDFSNF

SEQ ID NO: 32 - protein, 7 aa, synthetic construct
GYTFSNF

SEQ ID NO: 33 - protein, 7 aa, synthetic construct
GGDFSNF

SEQ ID NO: 34 - protein, 7 aa, synthetic construct
GYDFSSY

SEQ ID NO: 35 - protein, 7 aa, synthetic construct
GYDFSSF

SEQ ID NO: 36 - protein, 7 aa, synthetic construct
GYDFSNY

SEQ ID NO: 37 - protein, 6 aa, synthetic construct
YPGDGE

SEQ ID NO: 38 - protein, 6 aa, synthetic construct
YPIDGE

SEQ ID NO: 39 - protein, 6 aa, synthetic construct
YPGFGE

SEQ ID NO: 40 - protein, 11 aa, synthetic construct
RASGNIHNYLA

SEQ ID NO: 41 - protein, 11 aa, synthetic construct
RASGGIHNYLA

SEQ ID NO: 42 - protein, 11 aa, synthetic construct
RASQNIHNYLA

SEQ ID NO: 43 - protein, 11 aa, synthetic construct
RASGNISNYLA

SEQ ID NO: 44 - protein, 11 aa, synthetic construct
RASGNIHNSLA

SEQ ID NO: 45 - protein, 7 aa, synthetic construct
SAKRLES

SEQ ID NO: 46 - protein, 7 aa, synthetic construct
AASRLES

SEQ ID NO: 47 - protein, 7 aa, synthetic construct
SASRLES

SEQ ID NO: 48 - protein, 7 aa, synthetic construct
SAKRLAS

SEQ ID NO: 49 - protein, 7 aa, synthetic construct
SAKRLED

SEQ ID NO: 50 - protein, 9 aa, synthetic construct
QHFWSSPYT

SEQ ID NO: 51 - protein, 9 aa, synthetic construct
DDYLRAMDY

SEQ ID NO: 52 - protein, 9 aa, synthetic construct
DDYLRAMDI

SEQ ID NO: 53 - protein, 8 aa, synthetic construct
GYTFTDYE

SEQ ID NO: 54 - protein, 8 aa, synthetic construct
DYTFTDYY

SEQ ID NO: 55 - protein, 8 aa, synthetic construct
INPEAGGT

SEQ ID NO: 56 - protein, 8 aa, synthetic construct
INPESGGT

SEQ ID NO: 57 - protein, 8 aa, synthetic construct
INPNSGGT

SEQ ID NO: 58 - protein, 8 aa, synthetic construct
INPNAGGT

SEQ ID NO: 59 - protein, 8 aa, synthetic construct
IDPNSGGT

SEQ ID NO: 60 - protein, 8 aa, synthetic construct
IDPNAGGT

SEQ ID NO: 61 - protein, 12 aa, synthetic construct
QSVLYSSNNKNY

SEQ ID NO: 62 - protein, 6 aa, synthetic construct
HHHHHH

(19) United States
(12) Patent Application Publication
(10) Pub. No.: US 2025/0042996 A1
(43) Pub. Date: Feb. 6, 2025

(54) ANTIBODIES TO ANTI-SIGLEC-9 AND MULTISPECIFIC ANTIBODIES TARGETING SIGLEC-7 AND SIGLEC-9

(71) Applicants: Christopher BEBBINGTON, Redwood City, CA (US); Nenad TOMASEVIC, Redwood City, CA (US); Kenneth LUEHRSEN, Redwood City, CA (US); Thuy LUU, Redwood City, CA (US); Allakos, Inc., Redwood City, CA (US)

(72) Inventors: Wouter Korver, Redwood City, CA (US); Julia Schanin, Redwood City, CA (US); John Leung, Redwood City, CA (US); Bradford Youngblood, Redwood City, CA (US); Christopher Bebbington, Redwood City, CA (US); Nenad Tomasevic, Redwood City, CA (US); Kenneth Luehrsen, Redwood City, CA (US); Thuy Luu, Redwood City, CA (US)

(21) Appl. No.: 18/710,828

(22) PCT Filed: Nov. 16, 2022
(86) PCT No.: PCT/US2022/050144

§ 371 Date: May 16, 2024

Related U.S. Application Data
(60) Provisional application No. 63/280,000, filed on Nov. 16, 2021.

(51) Int. Cl. C07K 16/28; A61P 37/06

(52) U.S. Cl. CPC C07K 16/2803; A61P 37/06; C07K 2317/24; C07K 2317/31; C07K 2317/52; C07K 2317/565; C07K 2317/76; C07K 2317/77; C07K 2317/92

(57) ABSTRACT

The disclosure provides anti-Siglec-9 antibodies and bispecific and multispecific antibodies that target Siglec-9 and Siglec-7, and therapeutic methods comprising administering such antibodies to a cancer patient or a patient having an autoimmune disease or other disorder in which it is desirable to inhibit an immune response.

### CROSS-REFERENCE TO RELATED APPLICATIONS

This application claims priority benefit of U.S. Provisional Application No. 63/280,000, filed Nov. 16, 2021, which is incorporated by reference in its entirety for all purposes.

### BACKGROUND

Sialic Acid Binding Ig-Like Lectin 7 (Siglec-7), which is also known as p75 or AIRM; and Sialic Acid Binding Ig-Like Lectin 9 (Siglec-9), also known as CD329 or FOAP-9, are members of the sialic acid-binding lectins of the immunoglobulin (Ig) superfamily. Siglec receptors bind glycans containing sialic acid, but differ in their recognition of the linkage regiochemistry and spatial distribution of sialic residues. The members of the family also have distinct expression patterns. High level expression of Siglec-7 has been observed on Natural Killer (NK) cells. Expression has also been observed on a subset CD8+ T cells. Expression of Siglec-9 has been observed on Natural Killer (NK) cells, dendritic cells (DCs), macrophages, and monocytes.

Siglec-7 and Siglec-9 are inhibitory receptors that, when bound by ligand, inhibit immune system cells, e.g., NK cell and T cell, activity. Siglec-7 and Siglec-9 can be targeted for the treatment of cancer, e.g., using a neutralizing antibody that has reduced FcR binding and/or cross-linking activity, e.g., relative to an antibody that has a wildtype human IgG1 Fc region; or can be targeted for the treatment of inflammatory conditions, e.g., using an agonist antibody that has FcR binding and/or cross-linking activity.

### BRIEF SUMMARY

It is understood that this summary features various aspects of the disclosure and is not provided as a comprehensive summary of all embodiments encompassed by the disclosure.

In some aspects, the disclosure features bispecific and multispecific antibodies that comprise a Siglec-7 binding moiety and a Siglec-9 binding moiety. In some embodiments, the Siglec-9 binding moiety comprises an anti-Siglec 9 binding domain that binds to human Siglec-9 at a site for which the residue K123 of human Siglec-9 as determined with reference to SEQ ID NO:16 is important for binding to Siglec-9, but substitution of any one of residues L22, H48, W50, 151, or Y52 with alanine (A) has no effect on binding. In some embodiments, the Siglec-9 binding domain comprises a V_{H} region that comprises a CDR1 comprising DYTFT(G/D)YE (SEQ ID NO:21), a CDR2 comprising IDPEAGGT (SEQ ID NO:12) and a CDR3 comprising TRVWLH (SEQ ID NO:13); and a V_{L} region comprising a CDR1 comprising QSVLYSSNQKNY (SEQ ID NO:14), a CDR2 comprising WAS, and a CDR3 comprising HQYLSSNT (SEQ ID NO:8). In some embodiments, the V_{H} CDR1 comprises DYTFTDYE. In some embodiments, the Siglec-9 binding domain is a variant comprising at least one CDR in which one or two amino acids are substituted.

In some embodiments, a bispecific or multispecific antibody of the present disclosure comprises a Siglec-9 binding domain comprising a V_{H} region having least 90% identity to amino acid sequence SEQ ID NO:17 or 19, and a V_{L} region having at least 90% identity to amino acid sequence SEQ ID NO:18. In some embodiments, the Siglec-9 binding domain comprises a V_{H} region comprising amino acid sequence SEQ ID NO:17 or 19, and a V_{L} region comprising amino acid sequence SEQ ID NO:18. In some embodiments, the Siglec-9 binding domain comprises a V_{H} region comprising amino acid sequence SEQ ID NO:17 and a V_{L}region comprising amino acid sequence SEQ ID NO:18.

In some embodiments, a bispecific or multispecific antibody the present disclosure comprises a Siglec-9 binding domain comprising a V_{H} region having at least 90% identity to SEQ ID NO:17, in which variation in the amino acid sequence relative to SEQ ID NO:17 are in the framework region and a V_{L} region having at least 90% identity to SEQ ID NO:18 in which variations in the amino acid sequence relative to SEQ ID NO:18 are in the framework region.

In some embodiments, a bispecific or multispecific antibody the present disclosure comprises a Siglec-9 binding domain comprising a V_{H} region having at least 90% identity to SEQ ID NO:19, in which variation in the amino acid sequence relative to SEQ ID NO:19 are in the framework region and a V_{L} region having at least 90% identity to SEQ ID NO:18 in which variations in the amino acid sequence relative to SEQ ID NO:18 are in the framework region.

In some embodiments, a bispecific or multispecific antibody of the present disclosure comprises a Siglec-7 binding domain comprising: a V_{H} CDR1 comprising GYDFSNF, GYTFSNF, GGDFSNF, GYDFSSY, GYDFSSF, or GYDFSNY; a V_{H} CDR2 comprising YPGDGE, YPIDGE, or YPGFGE; a V_{H} CDR3 comprising DDYLRAMD(Y/V/I); a V_{L} CDR1 comprising RASGNIHNYLA, RASGGIHNYLA, RASQNIHNYLA, RASGNISNYLA, RASGNIHNSLA, or RASGNISNYLA; a V_{L} CDR2 comprising SAKRLES, AASRLES, SASRLES, SAKRLAS, or SAKRLED; and a V_{L} CDR3 comprising QHFWSSPYT; or a variant of the Siglec-7 binding domain in which one or more of the CDRs comprises one or two amino acid subsitutions. In some embodiments, the Siglec-7 binding domain comprises: a V_{H} CDR1 comprising GYDFSNF or GYDFSNY; a V_{H} CDR2 comprising YPGDGE; a V_{H}CDR3 comprising DDYLRAMD(Y/V/I); a V_{L} CDR1 comprising RASGNIHNYLA or RASQNIHNYLA; a V_{L} CDR2 comprising SAKRLES or SAKRLED; and a V_{L} CDR3 comprising QHFWSSPYT. In some embodiments, the Siglec-7 binding domain comprises a V_{H} CDR1 comprising GYDFSNF or GYDFSNY, a V_{H} CDR2 comprising YPGDGE; a V_{H}CDR3 comprising DDYLRAMDY or DDYLRAMDI, a V_{L} CDR1 comprising RASGNIHNYLA or RASQNIHNYLA, a V_{L} CDR2 comprising SAKRLES, and a V_{L} CDR3 comprising QHFWSSPYT. In some embodiments, the Siglec-7 binding domain comprises a V_{H} CDR1 comprising GYDFSNY, a V_{H} CDR2 comprising YPGDGE; a V_{H} CDR3 comprising DDYLRAMDY, a V_{L} CDR1 comprising RASQNIHNYLA, a V_{L} CDR2 comprising SAKRLES, and a V_{L} CDR3 comprising QHFWSSPYT.

In some embodiments, a bispecific or multispecific antibody of the present disclosure comprises a Siglec-7 binding domain comprising a V_{H} region having at least 90% identity to SEQ ID NO:6, 7, or 8 and a CDR1, CDR2 and CDR3 as shown in SEQ ID NOS:6, 7, or 8. In some embodiments, the Siglec-7 binding domain comprises a V_{H} of SEQ ID NO:6, 7, or 8. In some embodiments, the Siglec-7 binding domain comprises a V_{L} region having at least 90% identity to SEQ ID NO:9 or 10 and a CDR1, CDR2 and CDR3 as shown in SEQ ID NO:9 or 10. In some embodiments, the V_{L} region comprises SEQ ID NO:9 or 10.

In some embodiments, a bispecific or multispecific antibody the present disclosure comprises a Siglec-7 binding domain comprising a V_{H} region having at least 90% identity to SEQ ID NO:6 in which variation in the amino acid sequence relative to SEQ ID NO:6 are in the framework region and a V_{L} region having at least 90% identity to SEQ ID NO:9 in which variations in the amino acid sequence relative to SEQ ID NO:9 are in the framework region. In some embodiments, the Siglec-7 binding domain comprises a V_{H} region comprising SEQ ID NO:6 and a V_{L} region comprising SEQ ID NO:9.

In some embodiments, a bispecific or multispecific antibody of the present disclosure comprises a Siglec-9 binding domain comprising a V_{H} region comprising sequence SEQ ID NO:17 and a V_{L} region comprising SEQ ID NO:18; and a Siglec-7 binding domain that comprises a V_{H} region comprising SEQ ID NO:6 and a V_{L} region comprising SEQ ID NO:9.

In some embodiments, the bispecific or multispecific antibody comprises a monovalent binding fragment, e.g., an scFV or Fab fragment that binds to one of the targets of interest. In some embodiments the antibody comprises a human IgGFc region. In some embodiments, the human IgG Fc region comprises a mutation, relative to a native human IgG1 isotype sequence, that reduces binding of the antibody to an activating Fc receptor (FcR) and/or reduces Siglec receptor cross-linking. In some embodiments, the human IgG Fc region comprises a mutation, relative to a native human IgG1 isotype sequence, that enhances binding of the antibody to an Fc receptor (FcR) and/or enhance Siglec receptor cross-linking.

In a further aspect, the disclosure provides a pharmaceutical composition comprising a bispecific or multispecific antibody as described herein, e.g., in the preceding paragraphs in this section.

In an additional aspect, the disclosure provides a method of inhibiting cancer cell proliferation comprising administering a therapeutically effective amount of a bispecifc or multispecific antibody comprising a Siglec-9 binding moiety comprising a V_{H} region and V_{L}region as described herein, e.g., in the preceding paragraphs in this section, and a Siglec-7 binding moiety comprising a V_{H} region and V_{L} regions as described herein to a subject that has a tumor comprising cancer cells and/or immune cells that express Siglec-7 and Siglec-9 ligands. In some embodiments, the antibody comprises a human IgGFc region. In some embodiments, the Fc region comprises a mutation, relative to a native human IgG1 isotype sequence, that reduces binding of the antibody to an activating Fc receptor (FcR) and/or reduces Siglec-7 cross-linking and Siglec-9 cross-linking.

In another aspect, the disclosure provide a method of inhibiting an immune response in a subject in need thereof, the method comprising administering a therapeutically effective amount of a bispecific or multispecific antibody comprising a Siglec-9 binding moiety comprising a V_{H} region and V_{L} region as described herein, e.g., in the preceding paragraphs in this section, and a Siglec-7 binding moiety comprising a V_{H} region and V_{L} region as described herein to a subject, e.g., a subject that has an inflammatory or autoimmune disease. In some embodiments, the antibody comprises a human IgGFc region. In some embodiments, the Fc region comprises a mutation, relative to a native human IgG1 isotype sequence, that enhances binding of the antibody to an Fc receptor (FcR) and/or enhances Siglec-7 cross-linking and Siglec-9 cross-linking. In some embodiments, the subject has an autoimmune disease.

In some aspects, the disclosure features anti-Siglec-9 antibodies, compositions, and methods of using such antibodies for inhibiting tumor growth or for treatment of inflammatory diseases, such as autoimmune diseases, or other conditions in which it is desired to inhibit an immune response, e.g., treatment or prevention of transplant rejection.

Thus, in a further aspect, the disclosure provides an anti-Siglec-9 antibody that binds to an epitope in which residue K123 of human Siglec-9 as determined with reference to SEQ ID NO:16 is an important residue for binding to Siglec-9, but substitution of any one of residues L22, H48, W50, I51, or Y52 with alanine (A) has no effect on binding, wherein the anti-Siglec-9 antibody comprises a V_{H} region comprising a CDR1 sequence DYTFT(G/D)YE (SEQ ID NO:21), a CDR2 sequence IDPEAGGT (SEQ ID NO:12) and a CDR3 sequence TRVWLH (SEQ ID NO:13); and a V_{L} region comprising a sequence QSVLYSSNQKNY (SEQ ID NO:14), a CDR2 sequence WAS, and a CDR3 sequence HQYLSSNT (SEQ ID NO:8). In some embodiments, the the CDR1 sequence comprisesDYTFTDYE (SEQ ID NO:11). In some embodiments, the antibody comprises a V_{H} region having least 90% identity to amino acid sequence SEQ ID NO:17 or 19, and a V_{L} region having at least 90% identity to amino acid sequence SEQ ID NO:18, in which the changes in the amino acid sequence of the V_{H} region relative to SEQ ID NO:17 or 19 occur in the framework region and the changes in the V_{L} region relative to SEQ ID NO:18 occur in the framework region. In some embodiments, the antibody comprises a V_{H} region comprising amino acid sequence SEQ ID NO:17 or 19 and a V_{L} region comprising amino acid sequence SEQ ID NO:18. In some embodiments, the antibody is in a monovalent form. In some embodiments, the antibody is a multivalentFab form. In some embodiments the antibody comprises a human IgG Fc region.

In some embodiments, the human IgG Fc region comprises a mutation, relative to a native human IgG1 isotype sequence, that reduces binding of the antibody to an activatingFc receptor (FcR) and/or reduces Siglec-9 cross-linking. In some embodiments, the human IgG Fc region comprises a mutation, relative to a native human IgG1 isotype sequence, that enhances binding of the antibody to an Fc receptor (FcR) and/or enhance Siglec-9 cross-linking.

In an additional aspect, the disclosure provides a method of inhibiting cancer cell proliferation comprising administering a therapeutically effective amount of a neutralizing anti-Siglec-9 antibody having a V_{H} region and V_{L} region as described herein, e.g., in the preceding paragraphs in this section, or a bispecific or multispecific antibody comprising the antibody, to a subject that has a tumor comprising cancer cells and/or immune cells that express Siglec-9 ligands. In some embodiments, the antibody comprises a human IgG Fc region. In some embodiments, the Fc region comprises a mutation, relative to a native human IgG1 isotype sequence, that reduces binding of the antibody to an activating Fc receptor (FcR) and/or reduces Siglec-9 cross-linking.

In another aspect, the disclosure provide a method of inhibiting an immune response in a subject in need thereof, the method comprising administering a therapeutically effective amount of an antibody having a V_{H} region and V_{L} region as described herein, e.g., in the preceding paragraphs in this section, or a bispecific or multispecific antibody comprising the antibody, to the subject. In some embodiments, the antibody comprises a human IgG Fc region. In some embodiments, the Fc region comprises a mutation, relative to a native human IgG1 isotype sequence,, that enhances binding of the antibody to an Fc receptor (FcR) and/or enhance Siglec-9 cross-linking. In some embodiments, the subject has an autoimmune disease.

In some embodiments, an anti-Siglec-9 antibody of the present disclosure binds to an epitope in which residue K123 of human Siglec-9 as determined with reference to SEQ ID NO:16 is an important residue for binding to Siglec-9, but substitution of any one of residues L22, H48, W50, 151, or Y52 with alanine (A) has no effect on binding. In some embodiments, the antibody has a bivalent avidity of 50 pM or less. In some embodiments, the antibody blocks binding of ligand to Siglec-9, e.g., at an IC₅₀ of less than about 7 nM performed on T47D cells; and/or displaces ligand binding to Siglec-9 at an IC₅₀ of less than about 4 nM. In some embodiments, the anti-Siglec-9 antibody has an internalization activity of less than about 300 nM as measured using CD14+ monocytes from PBMC. In some embodiments, the antibody competes with an antibody having the six CDRs of an antibody designated herein as 9A12 (see, SEQ ID NO:1 and SEQ ID NO:2) for binding to Siglec-9. In some embodiments, the anti-Siglec-9 antibody has a V_{H} region that comprises at least one CDR, or at least two CDRs, of a heavy chain variable region sequence of SEQ ID NO:1. In some embodiments, the anti-Siglec-9 antibody has a V_{H} region that comprises a CDR3 of a heavy chain variable region sequence of SEQ ID NO:1. In some embodiments, the anti-Siglec-9 antibody has a V_{H} region that comprises a CDR1, CDR2, and CDR3 of a heavy chain variable region sequence of SEQ ID NO:1. In some embodiments, the anti-Siglec-9 antibody has a V_{L} region that comprises at least one CDR, or at least two CDRs, of a light chain variable region sequence of SEQ ID NO:2. In some embodiments, the anti-Siglec-9 antibody has a V_{L} region that comprises a CDR3 of a light chain variable region sequence of SEQ ID NO:2. In some embodiments, the anti-Siglec-9 antibody has a V_{L} region that comprises a CDR1, CDR2, and CDR3 of a light chain variable region sequence of SEQ ID NO:2. In some embodiments, the antibody comprises the six CDRs as shown in Table 1. In some embodiments, the antibody comprises one CDR, two CDRs, or three CDRs that differ form the corresponding CDR in Table 1 by one amino acid, or by two amino acids. In some embodiments, the antibody comprises at least one CDR that differs from a CDR as shown in Table 1 by three amino acids, or in some embodiments, by four amino acids.

In any of the foregoing embodiments, the antibody may be in a monovalent format or may be in an Fv, Fab, Fab′, scFv, diabody, or F(ab′)₂ fragment. In some embodiments, such an antibody is PEGylated.

In some embodiments, the anti-Siglec-9 antibody of the disclosure as described in the foregoing paragraphs is a multivalent form or bivalent form. In some embodiments, the antibody isn IgG, such as an IgG1, IgG2, IgG3, or IgG4.

In a further aspect, the disclosure provides a pharmaceutical composition comprising an anti-Siglec-9 antibody as described herein, e.g., in the preceding paragraph in this section, or a pharmaceutical composition comprising a bispecific or multispecific antibody comprising the anti-Siglec-9 antibody.

In an additional aspect, the disclosure provides a method of inhibiting proliferation of tumor cells, the method comprising administering a therapeutically effective amount of a neutralizing anti-Siglec-9 antibody as described herein, e.g., in the foregoing paragraphs, or a bispecific or multispecific antibody comprising such an antibody, to a patient that has a tumor that expresses Siglec-9 ligands. In some embodiments, the tumor expressed Siglec-9 ligands at a level higher than normal tissue.

In a further aspect, the disclosure provides a method of treating an immune disorder, such as an inflammatory disease or autoimmune disease, or a condition in which it is desirable to suppress an immune response, e.g., to treat or prevent transplant rejection, comprising administering an anti-Siglec-9 agonist antibody as described in the present disclosure.

### BRIEF DESCRIPTION OF THE DRAWINGS

FIG. 1 provides data from flow analysis of Siglec-9 recombinant protein binding or control to cancer cells from fresh primary tumors. +sia indicates sialidase stripped cells.

FIG. 2 provides data illustrating the percentage of Siglec-9-postive CD8+ T cells in various tumors

FIG. 3 provides illustrative data showing cross-reactivity, biochemical binding values, ligand blocking and displacement, and internalization of 12 illustrative Siglec-9 antibodies and a Siglec-9 antibody MAB1139 (R&D Systems).

FIG. 4 provides data illustrating ligand blocking activity of five anti-Siglec-9 antibodies and MAB1139.

FIG. 5 provides data illustrating ligand displacement activity of five anti-Siglec-9 antibodies and MAB1139.

FIG. 6 provides data illustrating internalization activity of twelve anti-Siglec-9 antibodies.

FIG. 7 provides data illustrating the effects of anti-Siglec-9 antibody on differentiation of dendritic cells (DCs).

FIG. 8 provides data illustrating the effects of anti-Siglec-9 antibody on DC activation markers.

FIG. 9 provides data illustrating the effects of anti-Siglec-9 antibody in a mixed lymphocyte reaction. Dendritic cells differentiated in the presence of anti-Siglec-9 mAb are more potent inducers of T cell proliferation than control treated DCs.

FIG. 10 provides data illustrating that DCs differentiated in the presence of anti-Siglec-9 antibody are more effective antigen presenting cells than DCs derived in the presence of control antibodies. Monocyte derived dendritic cells loaded with peptide are more effective at activating antigen specific T cells when differentiated in the presence of anti-Siglec-9 antibodies, resulting in higher percentages of activated T cells as measured by CD107a expression and levels of intracellular cytokines.

FIG. 11 provides data illustrating increased inflammatory cytokine production by primary monocytes from healthy donors differentiated into M1-type macrophages in the presence of anti-Siglec-9 antibody.

FIG. 12 provides data illustrating reduced production of immune-suppressive cytokines differentiated from primary monocytes from healthy donors differentiated into M2-type macrophages in the presence of anti-Siglec-9 antibody.

FIG. 13 provides data illustrating inhibition of the production of proinflammatory cytokines from CD16+ monocytes stimulated by LPS in the presence of an agonistic anti-Siglec-9 antibody.

FIG. 14 provides data illustrating inhibition of the production of proinflammatory cytokines from an autologous co-culture of CD16+ monocytes with T cells after T cell stimulation with CD3/CD28 in the presence of an agonistic anti-Siglec-9 antibody.

FIG. 15 illustrates reduced inflammation and disease activity by an agonistic anti-Siglec-9 antibody in an acute bleomycin-induced lung fibrosis model.

FIG. 16 summarizes illustrative antibody binding data. The antibody designated “9A12” competes with antibody K8 (described by Zhang et al (2000)) and has a distinct epitope, defined by critical residues, from known Siglec-9 antibodies Mab1139 (R&D Systems), E10⁻²⁸⁶ (BD Biosciences) and K8. 9A12 binding is affected by mutation at position K123. 1B6 and 12G10 compete with Mab1139 and have a distinct epitope from K8 and 9A12.

FIG. 17 summarizes antibodies that compete with one another for binding to Siglec-9 and summarizes residues of Siglec-9 that are important for antibody binding.

### DETAILED DESCRIPTION OF ASPECTS OF THE DISCLOSURE

### Terminology

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field. For example, for K_{D} and 1C₅₀ values±20%, ±10%, or ±5%, are within the intended meaning of the recited value.

Siglec-7, also known as p75 or AIRM, is a member of the sialic acid-binding lectins (Siglec) of the immunoglobulin (Ig) superfamily. Siglec receptors bind glycans containing sialic acid, but differ in their recognition of specific carbohydrate structures. A human Siglec-7 protein sequence available under accession number NP_055200.1 is as follows:

| | (SEQ ID NO: 20) |
|---|---|
| 1 | mllllllpll wgrervegqk snrkdysltm qssvtvqegm cvhvrcsfsy pvdsqtdsdp |
| 61 | vhgywfragn diswkapvat nnpawavqee trdrfhllgd pqtknctlsi rdarmsdagr |
| 121 | yffrmekgni kwnykydqls vnvtalthrp nilipgtles gcfqnltcsv pwaceqgtpp |
| 181 | miswmgtsvs plhpsttrss vltlipqpqh hgtsltcqvt lpgagvttnr tiqlnvsypp |
| 241 | qnltvtvfqg egtastalgn ssslsvlegq slrlvcavds npparlswtw rsltlypsqp |
| 301 | snplvlelqv hlgdegeftc raqnslgsqh vslnlslqqe ytgkmrpvsg vllgavggag |
| 361 | atalvflsfc vifivvrscr kksarpaadv gdigmkdant irgsasqgnl teswaddnpr |
| 421 | hhglaahssg eereiqyapl sfhkgepqdl sgqeatnney seikipk |

Siglec-9, also known as CD329 or FOAP-9, is a member of the sialic acid-binding lectins (Siglec) of the immunoglobulin (Ig) superfamily. Siglec receptors bind glycans containing sialic acid, but differ in their recognition of specific carbohydrate structures. A human Siglec-9 protein sequence available under Uniprotein accession number Q9Y336 is as follows:

(SEQ ID NO: 16)
MLLLLLPLLW GRERAEGQTS KLLTMQSSVT VQEGLCVHVP
CSFSYPSHGW IYPGPVVHGY WFREGANTDQ DAPVATNNPA
RAVWEETRDR FHLLGDPHTK NCTLSIRDAR RSDAGRYFFR
MEKGSIKWNY KHHRLSVNVT ALTHRPNILI PGTLESGCPQ
NLTCSVPWAC EQGTPPMISW IGTSVSPLDP STTRSSVLTL
IPQPQDHGTS LTCQVTFPGA SVTTNKTVHL NVSYPPQNLT
MTVFQGDGTV STVLGNGSSL SLPEGQSLRL VCAVDAVDSN
PPARLSLSWR GLTLCPSQPS NPGVLELPWV HLRDAAEFTC
RAQNPLGSQQ VYLNVSLQSK ATSGVTQGVV GGAGATALVF
LSFCVIFVVV RSCRKKSARP AAGVGDTGIE DANAVRGSAS
QGPLTEPWAE DSPPDQPPPA SARSSVGEGE LQYASLSFQM
VKPWDSRGQE ATDTEYSEIK IHR

The term “antibody” is used herein in the broadest sense and encompasses various antibody structures, including but not limited to monoclonal antibodies, polyclonal antibodies, multispecific antibodies, such as bispecific antibodies, and antibody fragments so long as they exhibit the desired antigen-binding activity. A binding domain in the context of an antibody refers to the region of the antibody that binds to the antigen, which in some embodiments may be a fragment comprising the antibody variable region.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules, such as scFv molecules. Reference to an “antibody” includes multispecific or bispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. The term “CDR” may be used interchangeably with “HVR”.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc,M.-P. IMGT, the international ImMunoGeneTics database. Nucleic Acids Res. Jan 1;29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272(1989); Martin, et al, Methods Enzymol., 203, 121-153, (1991); Pedersen et al, Immunomethods, 1, 126, (1992); and Rees et al, In Sternberg M.J.E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM, an Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region “as used herein includes naturally occurring allelic variants of the Fc region as well as modifications that modulate effector function, including modifications that decrease or increase binding of an Fc region to an Fc receptor, and modifications that influence antibody stability, e.g., increase serum half-life. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, etal., Science 247:306-1310, 1990). For example, for IgG4 antibodies, a single amino acid substitution (S228P according to Kabatnumbering; designated IgG4Pro) may be introduced to abolish the heterogeneity observed in recombinantIgG4 antibody (see, e.g., Angal, etal.,Mol Immunol30:105-108, 1993).

As used herein, an “agonist” antibody in the context of an anti-Siglec-9 antibody of the present disclosure refers to an antibody that binds to an epitope of Siglec-9 and activates the native inhibitory function of Siglec-9, e.g., by interacting with an Fc receptor via binding of an Fc region, thus leading to Siglec-9 cross-linking and inhibition of immune cell activity.

As used herein, an “agonist” antibody of the present disclosure in the context of a bispecific or multispecific antibody refers to an antibody that activates the native inhibitory function of Siglec receptors (i.e., Siglec-7 and Siglec-9), e.g., by interacting with an Fc receptor via binding of an Fc region, thus leading to Siglec receptor cross-linking and inhibition of immune cell activity.

As used herein, a “neutralizing” anti-Siglec-9 antibody refers to an antibody that blocks the inhibitory activity of Siglec-9, e.g., by internalizing Siglec-9 or blocking ligand interactions with Siglec-9. In some embodiments, a neutralizing antibody comprises an Fc region that does not bind and Fc receptor or exhibits reduced binding, e.g., a reduction by at least 50%, or at least 80%, or at least 90%, or greater binding to an Fc receptor compared to a native human IgG1 Fc region.

As used herein, a “neutralizing” bispecific or multispecific antibody refers to an antibody that blocks the inhibitory activity of Siglec receptors (i.e., Siglec-7 or Siglec-9) e.g., by internalizing receptor or blocking ligand interactions with the Siglect receptor. In some embodiments, a neutralizing antibody comprises an Fc region that does not bind an Fc receptor or exhibits reduced binding, e.g., a reduction by at least 50%, or at least 80%, or at least 90%, or greater binding to an Fc receptor compared to a native human IgG1 Fc region.

The term “equilibrium dissociation constant” abbreviated (K_{D}), refers to the dissociation rate constant (k_{d}, time⁻¹) divided by the association rate constant (kₐ, time⁻¹ M⁻¹).

Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵ M, less than 10⁻⁵ M, less than 5 ×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5 x10⁻¹⁰ M, less than 10⁻¹⁰ M, less than 5 ×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a bivalent antibody. In the context of the present disclosure, an “improved” K_{D} refers to a lower K_{D}. In some embodiments, an antibody of the present disclosure has a K_{D} of less than 5×10⁻⁵M, less than 10⁻⁵M, less than 5×10⁻⁶M, less than 10⁻⁶ M, less than 5×10⁻⁷M, less than 10⁻⁷M, less than 5×10⁻⁸M, less than 10⁻⁸M, less than 5×10-9 M, less than 10⁻⁹M, less than 5 x10⁻¹⁰ M, less than 10⁻¹⁰M, less than 5×10⁻¹¹ M, less than 10-¹¹M, less than 5×10⁻¹²M, less than 10⁻¹²M, less than 5×10⁻¹³M, less than 10⁻¹³M, less than 5 ×10⁻¹⁴M, less than 10⁻¹⁴M, less than 5×10⁻¹⁵M, or less than 10⁻¹⁵M or lower as measured as a monovalent antibody, typically a monovalent Fab. In some embodiments, an anti-Siglec-9 antibody or anti-Siglec-7 antibody as disclosed herein has K_{D} less than 100 pM, e.g., or less than 75 pM, e.g., in the range of 1 to 100 pM, when measured as a monovalent Fab by surface plasmon resonance analysis using a biosensor system such as a Biacore® system performed at 37° C. In some embodiments, an anti-Siglec-9 antibody or anti-Siglec-7 antibody has K_{D} less than 500 pM, e.g., in the range of 1 to 500 pM, or 1 to 200, or 1 to 250 pM, when measured as a monovalent Fab by surface plasmon resonance analysis using a biosensor system such as a Biacore® system performed at 37° C. In the context of the present disclosure, an “improved” K_{D} refers to a lower K_{D}.

The term “valency” as used herein refers to the number of different binding sites of an antibody for an antigen. A monovalent antibody comprises one binding site for an antigen. A bivalent antibody comprises two binding sites for the same antigen.

The term “monovalent molecule” as used herein refers to a molecule that has one antigen-binding site, e.g., a Fab.

The term “bivalent molecule” as used herein refers to a molecule that has two antigen-binding sites. In some embodiments, a bivalent molecule of the present disclosure is a bivalent antibody or a bivalent fragment thereof. In some embodiments, a bivalent molecule of the present disclosure is a bivalent antibody. In some embodiments, a bivalent molecule of the present disclosure is an IgG. In general monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. Preferably both antigen-binding sites of a bivalent molecule share the same antigen specificity.

A “bispecific” antibody as used herein refers to an antibody that has binding specificity for at least two different epitopes.

A “multispecific” antibody has used herein refers to an antibody that has binding specific for two or more different epitopes. A “bispecific” antibody is an example of a “multispecific” antibody.

The term “avidity” as used herein in the context of antibody binding to an antigen refers to the combined binding strength of multiple binding sites of the antibody. Thus, “bivalent avidity” refers to the combined strength of two binding sites.

The phrase “specifically (or selectively) binds” to an antigen or target or “specifically (or selectively) immunoreactive with,” when referring to a protein or peptide, refers to a binding reaction whereby the antibody binds to the antigen or target of interes, typically with a at least a 100-fold greater (stronger) affinity than other antigens. For example, in the context of this disclosure, an antibody, or Siglec-9 binding domain, binding to Siglec-9 with a K_{D} that is at least 100-fold greater than its affinity for other antigens.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, BLAST 2.0 can be used with the default parameters described to determine percent sequence.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

### Anti-Siglec-9 Antibodies

Neutralizing anti-Siglec-9 antibodies of the disclosure have improved binding characteristics compared to known anti-Siglec 9 antibodies, such as improved internalization activity and/or improved ligand-blocking or ligand displacement activity in combination with high affinity binding. Agonist antibodies of the disclosure have potent Fc receptor interaction to facilitate cross-linking of Siglec-9. A neutralizing anti-Siglec-9 antibody, i.e., that blocks Siglec-9 immune cell inhibitory activity, in accordance with the disclosure can be used for the treatment of cancer. An agonist anti-Siglec-9, i.e., that activates Siglec-9 inhibitory activity, can be used for the treatment of autoimmune and other inflammatory diseases. Thus, in some embodiments, an anti-Siglec-9 antibody of the present disclosure comprises an Fc region that does not bind to Fc receptors, or exhibits reduced binding, e.g., by at least 70% or greater, to an Fc receptor compared to the native human IgG Fc region isotype and/or does not cross-link anti-Siglec-9. In other embodiments, an anti-Siglec-9 antibody of the present disclosure is an agonist antibody that comprises an Fc region that binds to Fc receptors, e.g., exhibits binding equivalent to that of a native Fc-receptor-binding Fc region, or stronger, thus, for example, cross-linking Siglec-9 and activating its inhibitory activity.

In some embodiments, an anti-Siglec-9 antibody of the present disclosure enhances dendritic cell activity, typically by enhancing T cell activation. In the context of the present disclosure, “enhancement” of dendritic cell activity includes increasing differentiation of monocytes into mature dendritic cells, increasing the level of dendritic cell activation markers expressed by the cells, and increasing the potential to stimulate proliferation in mixed lymphocyte reactions and in autologous peptide presentation and T cell activation assays. Dendritic cell activity is typically “enhanced” activity by at least 10%, at least 20%, at least 25%, at least 30%, at least 355%, at least 40%, at least 45%, at least 50%, at least 60%, at least 70%, at least 80%, at least 90%, or greater, with treatment with an anti-Siglec-9 antibody as compared to controls using an isotype-matched control antibody. Often, “enhancement” is determined by assessing T cell activation and T-cell proliferation. Illustrative assays are provided in the examples section.

In some embodiments, an agonistic anti-Siglec-9 antibody of the present disclosure inhibits the production of inflammatory cytokines from LPS activated CD16+ monocytes.

In some embodiments, an agonistic anti-Siglec-9 antibody of the present disclosure reduces monocytes frequencies and inhibits fibrosis in a bleomycin induced lung fibrosis model.

In some embodiments, an agonistic anti-Siglec-9 antibody of the present disclosure inhibits the production of inflammatory cytokines from T cells co-cultured with autologous CD16+ monocytes after stimulation with CD3/CD28.

In some embodiments, an anti-Siglec-9 antibody of the present disclosure has a K_{D}(bivalent avidity) of less than about 200 pM, or less than about 75 pM, or less than about 75 pM. In some embodiment has a K_{D} (bivalent avidity) of 50 pM or less when measured in an IgG format. In some embodiments, the anti-Siglec-9 antibody has a K_{D} (bivalent avidity) of less than about 40 pM or less than about 20 pM when measured in an IgG format. In some embodiments, an antibody of the disclosure has a K_{D} (bivalent avaidity) of about 1 pM or less when measured in an IgG format. In some embodiments, an antibody of the disclosure has at least one, at least two, or three heavy chain CDRs as shown in Table 1 and at least one, at least two, or three light chain CDRs V_{L} sequence shown in Table 1.

In some embodiments, an anti-Siglec-9 antibody of the present disclosure blocks ligand binding to Siglec-9. In the context of the present disclosure, the ability to block ligand binding refers to the concentration of monoclonal antibody at which 50% of Siglec-9 does not bind ligand. Ligand blocking activity can be assessed using known assays. For example, ligand blocking activity may be determined using a cell line, such as the human breast cancer cell line T47D, that expresses high levels of ligands for Siglec-9 on the cell surface. IC₅₀ values for blocking can be determined, for example, as explained in the examples section. In some embodiments, an antibody of the disclosure that has a ligand blocking activity IC₅₀ of less than 7000 pM, or less than about 4000 pM or less than about 3000 pM or less than about 2000 pM or less than about 1500 pM or less than about 1000 pM, when assayed under the assay conditions described in the Examples section. In some embodiments, an antibody of the disclosure has at least one, at least two, or three heavy chain CDRs as shown in Table 1 and at least one, at least two, or three light chain CDRs V_{L} sequence shown in Table 1.

In some embodiments, an anti-Siglec-9 antibody of the present disclosure displaces ligand, e.g., at an IC₅₀ of less than about 4 nM. In the context of the present disclosure, the ability to displace ligand refers to the concentration of monoclonal antibody at which 50% of ligand binding to Siglec-9 is displaced. Ligand displacement activity can be assessed using known assays. For example, ligand displacement activity may be determined using a cell line, such as the human breast cancer cell line T47D, that expresses high levels of ligands for Siglec-9 on the cell surface. IC₅₀ values for displacement can be determined, for example, as explained in the examples section. In some embodiments, an antibody of the disclosure that has a ligand displacing activity IC₅₀ of less than 4000 pM, or less than about about 3000 pM or less than about 2000 pM or less than about 1500 pM or less than about 1000 pM, when assayed under the assay conditions described in the Examples section. In some embodiments, an antibody of the present disclosure that exhibits ligand displacing activity has at least one, at least two, or three heavy chain CDRs as shown in Table 1 and at least one, at least two, or three light chain CDRs V_{L} sequence shown in Table 1.

In some embodiments, an anti-Siglec-9 antibody of the present disclosure exhibits internalization activity. In the present disclosure, internalization activity refers to the concentration of antibody at which 50% of Siglec-9 is internalized in 24 hours on healthy CD14+peripheral blood mononuclear cells (PBMC). Internalization can be assessed using known assays. For example, PBMC obtained from healthy donors may be used to determine internalization activity of anti-Siglec-9 antibodies. IC₅₀ values for internalization can be determined, for example, as described in the Examples section. In some embodiments, an antibody of the has an internalizing IC₅₀ of less than about 300 nM. In some embodiments, an antibody has an internalizing IC₅₀ of less than about 200 nM, or less than about 150 nM, or less than about 100 nM, when assayed under the assay conditions described in the Examples section. In some embodiments, an antibody of the present disclosure that exhibits internalization activity has has at least one, at least two, or three heavy chain CDRs as shown in Table 1 and at least one, at least two, or three light chain CDRs V_{L} sequence shown in Table 1.

In some embodiments, an anti-Siglec-9 antibody of the present disclosure binds to an epitope in which residues Y52 and/or K123 of human Siglec-9 as determined with reference to SEQ ID NO:16 are important residues for binding to Siglec-9, but substitution of any one of residues L22, H48, W50, 151, Y52, or K123 with Ala (A) has no effect on binding. In some embodiments, the antibody competes with an antibody having the HCDR1, HCDR2, HCDR3, LCDR1, LCDR2, and LCDR3 sequences as defined for an antibody as shown in Table 1 for binding to Siglec-9. In some embodiments, the antibody competes with an antibody comprising: (i) the V_{H} sequence of SEQ ID NO:1 and the V_{L} sequence of SEQ ID NO:2 for binding to Siglec-9. In some embodiments, the antibody comprises HCDR1, HCDR2, HCDR3, LCDR1, LCDR2, and LCDR3 sequences as defined in Table 1.

In some embodiments, an anti-Siglec-9 antibody of the disclosure comprises at least one, two, or all three CDRs of a heavy chain variable amino acid sequence set forth in Table 1 as defined by Kabat; and/or comprises at least one, two, or all three CDRs of a light chain variable amino acid sequence set forth in Table 1 as defined by Kabat. In some embodiments, the antibody comprises the HCDR1, HCDR2, HCDR3, LCDR1, LCDR2, or LCDR3 as shown in Table 1 as defined by Kabat.

In some embodiments, an anti-Siglec-9 antibody of the disclosure comprises at least one, two, or all three CDRs of a heavy chain variable amino acid sequence set forth in Table 1 as defined by Chothia; and or comprises at least one, two, or all three CDRs of a light chain variable amino acid sequence set forth in Table 1 as defined by Chothia. In some embodiments, the antibody comprises the HCDR1, HCDR2, HCDR3, LCDR1, LCDR2, or LCDR3 as shown in Table 1 as defined by Chothia.

In some embodiments, an anti-Siglec-9 antibody of the disclosure comprises at least one, two, or all three CDRs of a heavy chain variable amino acid sequence set forth in Table 1 as defined by IMGT; and/or comprises at least one, two, or all three CDRs of a light chain variable amino acid sequence set forth in Table 1 as defined by IMGT. In some embodiments, the antibody comprises the HCDR1, HCDR2, HCDR3, LCDR1, LCDR2, or LCDR3 as shown in Table 1 as defined by IMGT.

In some embodiments, an anti-Siglec-9 antibody comprises a V_{H}that comprises a CDR3 sequence comprising SEQ ID NO:5. In some embodiments, V_{H} region has a variant CDR3 sequence comprising SEQ ID NO:5 in which one amino acid residue is substituted. In some embodiments, the antibody further comprises a V_{H} CDR1 sequence of SEQ ID NO:3 or SEQ ID NO:9; and/or a V_{H} CDR2 sequence of SEQ ID NO:4 or SEQ ID NO:10. In some embodiments, the antibody further comprises a V_{H} CDR1 sequence of SEQ ID NO:3 or SEQ ID NO:9 in which one or two amino acids are substituted; and/or a V_{H} CDR2 sequence of SEQ ID NO:4 or SEQ ID NO:10 in which one or two amino acids are substituted. In some embodiments, the anti-Siglec-9 antibody further comprises a V_{L} comprising a CDR3 sequence that comprises SEQ ID NO:8 or a variant thereof in which one amino acid residue is substituted. In some embodiments, the antibody further comprises a V_{L} CDR1 sequence of SEQ ID NO:6; and/or a V_{L} CDR2 sequence of SEQ ID NO:7. In some embodiments, the antibody further comprises a V_{L} CDR1 sequence of SEQ ID NO:6 in which one or two amino acids are substituted; and/or a V_{L} CDR2 sequence of SEQ ID NO:7 in which one amino acid is substituted.

In some embodiments, an anti-Siglec-9 antibody comprises a V_{H}that comprises a CDR3 sequence comprising SEQ ID NO:13. In some embodiments, V_{H} region has a variant CDR3 sequence comprising SEQ ID NO:13 in which one amino acid residue is substituted. In some embodiments, the antibody further comprises a V_{H} CDR1 sequence of SEQ ID NO:11 and/or a V_{H} CDR2 sequence of SEQ ID NO:12. In some embodiments, the antibody further comprises a V_{H} CDR1 sequence of SEQ ID NO:11 in which one or two amino acids are substituted; and/or a V_{H} CDR2 sequence of SEQ ID NO:12 in which one or two amino acids are substituted. In some embodiments, the anti-Siglec-9 antibody further comprises a V_{L}comprising a CDR3 sequence that comprises SEQ ID NO:8 or a variant thereof in which one amino acid residue is substituted. In some embodiments, the antibody further comprises a V_{L}CDR1 sequence of SEQ ID NO:14 and/or a V_{L} CDR2 sequence WAS. In some embodiments, the antibody further comprises a V_{L} CDR1 sequence of SEQ ID NO:14 in which one or two amino acids are substituted; and/or a V_{L} CDR2 sequence WAS. In some embodiments, the antibody comprises an HCDR1 sequence of SEQ ID NO:11 or DYTFTGYE (SEQ ID NO:15); and HCDR2 sequence of SEQ ID NO:12, an HCDR3 sequence of SEQ ID NO:13 and LCDR1 sequence of SEQ ID NO:14, and LCDR2 sequence WAS, and an LCDR3 sequence of SEQ ID NO:8.

TABLE 1

| V_{H} CDR | CDR1 | CDR2 | CDR3 |
|---|---|---|---|
| (definition) | (SEQ ID NO) | (SEQ ID NO) | (SEQ ID NO) |
| Kabat | DYEMH | AIDPEAGGTDYNQKFKG | VWLH |
| | (SEQ ID NO: 3) | (SEQ ID NO: 4) | (SEQ ID NO: 5) |
| Chothia | DYTFTDY | DPEAGG | VWLH |
| | (SEQ ID NO: 9) | SEQ ID NO: 10) | (SEQ ID NO: 5) |
| IMGT | DYTFTDYE | IDPEAGGT | TRVWLH |
| | (SEQ ID NO: 11) | (SEQ ID NO: 12) | (SEQ ID NO: 13) |
| V_{L} CDR | CDR1 | CDR2 | CDR3 |
| (CDR definition) | (SEQ ID NO) | (SEQ ID NO) | (SEQ ID NO) |
| Kabat/Chothia | KSSQSVLYSSNQKNYLA | WASTRES | HQYLSSNT |
| | (SEQ ID NO: 6) | (SEQ ID NO: 7) | (SEQ ID NO: 8) |
| IMGT | QSVLYSSNQKNY | WAS | HQYLSSNT |
| | (SEQ ID NO: 14) | | (SEQ ID NO: 8) |

In some embodiments, an anti-Siglec-9 antibody of the present disclosure comprises a heavy chain variable region having at least 7000, 7500, 800%,8500,9000,9100,9200,9300 5 94%, 95%, 96%, 97%, ⁹⁸%, 99%, or 100% sequence identity to the amino acid sequence of a heavy chain variable region SEQ ID NO:1 and a light chain variable region of SEQ ID NO:2, where the light chain variable region has at least 700%, 7500 8000, 8500, 9000, 9100, 9200, 9300 94%, 95%, 96%, 97%, 98%, 99%, or 100% sequence identity to the amino acid sequence of the light chain variable region of SEQ ID NO:2. In some embodiments, the antibody comprises CDRs as defined by Kabat and modifications to the heavy chain variable region and/or the light chain variable region in the framework region compared to SEQ ID NO:1 (V_{H}) or SEQ ID NO:2 (V_{L}).

In some embodiments, an anti-Siglec-9 antibody of the present disclosure comprises a heavy chain variable region having at least 70%, 7500 8000,8500,9000,9100,9200,9300 94%, 95%, 96%, 97%, 98%, 99%, or 100% sequence identity to the amino acid sequence of a heavy chain variable region SEQ TD NO:1 and a light chain variable region of SEQ ID NO:2, where the light chain variable region has at least 70%, 7500 80%, 8500, 9000, 9100, 9200, 9300 94%, 95%, 96%, 97%, 98%, 99%, or 100% sequence identity to the amino acid sequence of the light chain variable region of SEQ ID NO:2. In some embodiments, the antibody comprises CDRs as defined by Chothia and modifications to the heavy chain variable region and/or the light chain variable region in the framework region compared to SEQ ID NO:1 (V_{H}) or SEQ ID NO:2 (V_{L}).

In some embodiments, an anti-Siglec-9 antibody of the present disclosure comprises a heavy chain variable region having at least 70%, 75%, 80%, 85%, 90%, 91%, 92%, 93%, 94%, 95%, 96%, 97%, 98%, 99%, or 100% sequence identity to the amino acid sequence of a heavy chain variable region SEQ ID NO:1 and a light chain variable region of SEQ ID NO:2, where the light chain variable region has at least 70%, 75%, 80%, 85%, 90%, 91%, 92%, 93%, 94%, 95%, 96%, 97%, 98%, 99%, or 100% sequence identity to the amino acid sequence of the light chain variable region of SEQ ID NO:2. In some embodiments, the antibody comprises CDRs as defined by IMGT and modifications to the heavy chain variable region and/or the light chain variable region in the framework region compared to SEQ ID NO:1 (V_{H}) or SEQ IS NO:2 (V_{L}).

In a further aspect of the disclosure, an anti-Siglec-9 antibody according to any of the above embodiments is a monoclonal antibody, including a chimeric, humanized or human antibody. In one embodiment, an anti-Siglec-9 antibody is an antibody fragment, e.g., a Fv, Fab, Fab′, scFv, diabody, o rF(ab′)₂fragment. In another embodiment, the antibody is a substantially full length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells (e.g. E. coli o rphage), as described herein.

In some embodiments an anti-Siglec-9 antibody in accordance with the present disclosure is a ligand blocking antibody in a monovalent format. In some embodiments, the anti-Siglec-9 antibody is in a fragment format, e.g., a Fv, Fab, Fab′, scFv, diabody, or F(ab′)₂ fragment. In some embodiments, an antibody in a monovalent or fragment format is modified, for example by PEGylation, to extend half-life.

In some embodiments, an anti-Siglec-9 antibody of the present disclosure is employed in a bispecific or multispecific format. For example, in some embodiments, the antibody may be incorporated into a bispecific or multispecific antibody that comprises a therapeutic antibody, as further discussed below.

### Fc Engineering

An anti-Siglec-9 antibody of the present disclosure can comprise an Fc region, which as described herein, may be a variant Fc region engineered to alter one or more functional properties of the antibody, such as extending serum half-life and/or reducing or enhancing effector function, including reducing complement fixation, Fc receptor binding, and/or antibody-dependent cell-mediated cytotoxicity. An anti-Siglec-9 antibody of the present disclosure may thus have one or more Fc mutations as specifically detailed below in the section relating to Fc engineering of bispecific and multispecific antibodies.

### Production of Anti-Siglec-9 Antibodies

Antibodies can be produced using any number of expression systems, including prokaryotic and eukaryotic expression systems (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. In some embodiments, the expression system is a mammalian cell expression, such as a CHO cell expression system. In embodiments in which an antibody comprises both a V_{H} and V_{L} region, the V_{H} and V_{L} regions may be expressed using a single vector, e.g., in a bicistronic expression unit, or under the control of different promoters. In other embodiments, the V_{H} and V_{L} region may be expressed using separate vectors. A V_{H} or V_{L} region as described herein may optionally comprise a methionine at the N-terminus. Any suitable expression vector may be used, including plasmid vectors and viral vectors such as adenoviruses, adeno-associated viruses, retroviruses, lentivirus, or other viral vectors.

In some embodiments, vertebrate host cells are used for producing an anti-Siglec-9 antibody of the present disclosure. Illustrative mammalian cell lines for production of antibody heavy and light chains include, but are not limited to, CV1, BHK, TM4, VERO, MDCK, HepG2, and CHO cells.

A host cell transfected with one or more expression vectors encoding an anti-Siglec-9 antibody heavy and/or light chain of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

### Bispecific and Multispecific Antibodies

Bispecific or multispecific antibodies are antibodies that have binding specificities for at least two different epitopes. In the context of the present disclosure, a bispecific or multispecific antibody comprises a Siglec-7 binding moiety that specifically bind Siglec-7 and and a Siglec-9 binding moiety that specifically binds Siglec-9. Such antibodies can be derived from full length antibodies or antibody fragments (e.g., F(ab′)₂ bispecific antibodies).

Generally, bispecific antibodies may be produced in a variety of molecular formats as reviewed by Brinkmann et al., Mabs 9:182-212, 2017. See also, Wang etal, Antibodies (Basel) 8:43, 2019; Liu et al., Front. Immunol 8:38, 2017. Bispecific or multispecific antibody-based molecules may also be produced by chemical conjugation or coupling of individual full length IgGs or coupling of fragments of IgGs to form multispecific and multivalent antibody derivatives. Examples are chemically coupled Fab fragments, IgG-dimers, etc. In some embodiments, multispecific molecules may also be produced by combining recombinant and chemical methods.

In some embodiments, antibody variable domains with the desired binding specificities (antibody-antigen combining sites) are fused to immunoglobulin constant domain sequences. The fusion is typically to an immunoglobulin heavy chain constant domain, comprising at least part of the hinge, CH2, and CH3 regions.

Techniques for making bispecific or multispecific antibodies include, but are not limited to, recombinant co-expression of two immunoglobulin heavy chain-light chain pairs having different specificities (see Milstein and Cuello, Nature 305: 537 (1983)) and “knob-in hole” engineering (see, e.g., U.S. Pat. No. 5,731,168, and Atwell et ah, J. Mol. Biol. 270:26 (1997)). Nonlimiting exemplary knob-in-hole substitutions include T366W (knob) and T366S/L368A/Y407V (hole). In some embodiments, the knob-in-hole substitutions are in IgG1 constant domains

Multispecific or bispecific antibodies may also be made by engineering electrostatic steering effects for making antibody Fc-heterodimeric molecules. See, e.g., WO 2009/089004; Dillon et ah, Mabs 9(2): 213-230 (2017). As a nonlimiting example, in a bispecific antibody comprising two heavy chain variable regions and two light chain variable regions, a first heavy chain variable region may comprise a Q39E substitution (Kabatnumbering) and a first light chain variable region may comprise a Q38K substitution (Kabat numbering); and a second heavy chain variable region may comprise a Q39K substitution (Kabatnumbering) and a second light chain variable region may comprise a Q38E substitution (Kabat numbering). In some embodiments, the Q39E/Q38K and Q39K/Q38E substitutions reduce mispairing of the heavy and light chains of the bispecific antibody. Similarly, a first heavy chain constant region may comprise a S183K substitution (EU numbering) and a first light chain constant region may comprise a V133E substitution (EU numbering), and the a second heavy chain constant region may comprise a S183E substitution (EU numbering) and a second light chain constant region may comprise a V133K substitution (EUnumbering). In some embodiments, the S183K/V133E and S183E/V133K substitutions reduce mispairing of the heavy and light chains of the bispecific antibody.

In some embodiments, a bispecific antibody comprises Q39E/Q38K and Q39K/Q38E substitutions in the binding domains and S183K/V133E and S183E/V133K substitutions in the constant regions. In some embodiments, a bispecific antibody comprises both knob-in-hole substitutions and electrostatic substitutions. See, e.g., WO 2016/172485.

Various further molecular formats for multispecific antibodies are known in the art (see e.g., Spiess et al., Mol Immunol 67 (2015) 95-106). Multispecific antibodies may also be made by cross-linking two or more antibodies or fragments (see, e.g., U.S. Pat. No. 4,676,980, and Brennan et al., Science, 229: 81(1985)); using leucine zippers to produce bi-specific antibodies (see, e.g., Kostelny et al., J Immunol., 148(5): 1547-1553 (1992) and WO 2011/034605); using the common light chain technology for circumventing the light chain mis-pairing problem (see, e.g., WO 98/50431); using“diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruberet al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig may also be generated (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831.

Multispecific antibodies may also be provided in an asymmetric form with a domain crossover in one or more binding arms of the same antigen specificity, i.e. by exchanging the VH/VL domains (see e.g., WO 2009/080252 and WO 2015/150447), the CHI/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010⁻²⁰). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CHI), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Examples of bispecific antibody formats that can be employed include, but are not limited to, “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., ProtEng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) el203498. A bispecific antibody or antigen binding fragment thereof also includes a “Dual Acting FAb” or “DAF” (see, e.g., US 2008/0069820 and WO 2015/095539).

Bispecific and multispecific antibodies of the present disclosure are thus produced using methodology apparent to those of skill in the art.

### Siglec-9 Binding Moiety

A bispecific or multispecific antibody that targets Siglec-7 and Sigle-9 comprises any one of the anti-Siglec-9 antibody binding domains as detailed above in the section Anti-Siglec-9 Antibodies.

### Siglec-7 Binding Moiety

In some embodiments, a Siglec-7 binding domain comprises a heavy chain and light chain as described in U.S. Patent Application Publication No. 2019/0194323, which is incorporated by reference.

In some embodiments, a Siglec-7 binding domain of the present disclosure internalizes Siglec-7 and comprises an HCDR1, HCDR2, HCDR3 of SEQ ID NO:22, an LCDR1, LCDR2, LCDR3 of SEQ ID NO:23, or a variant thereof in which one, two, three, or four of the CDR2 comprises 1, 2, 3, or 4 substitutions relative to the corresponding CDR. In some embodiments, the antibody has at least one, at least two, or three CDRs of a V_{H} sequence as set forth in any one of SEQ ID NOS:6-8; and at least one, at least two, or three CDRs of a V_{L} sequence as set forth in SEQ ID NO:9 or 10.

In some embodiments, a Siglec-7 binding domain of the present disclosure comprises a heavy chain variable region having at least 80%, 85%, 90%, 91%, 92%, 93%, 94%, 95%, 96%, 97%, 98%, or 99% sequence identity to the amino acid sequence of a heavy chain variable region of any one of SEQ ID NOS:6-8 and comprises the three CDRs of any one of SEQ ID NOS:6-8, a variant of at least one of the three CDRs in which 1, 2, 3, or 4 amino acids are substituted. In certain embodiments, a V_{H} sequence having at least 80%, 85%, 90%, 91%, 92%, 93%, 94%, 95%, 96%, 97%, 98%, or 99% identity to the amino acid sequence of a heavy chain variable region of any one of SEQ ID NOS:6-8 contains substitutions (e.g., conservative substitutions), insertions, or deletions relative to the reference sequence, but the binding domain retains the ability to bind Siglec-7.

In some embodiments, a Siglec-7 binding domain of the present disclosure comprises a heavy chain variable region having at least 90%, 91%, 92%, 93%, 94%, 95%, 96%, 97%, 98%, or 99% sequence identity to the amino acid sequence of a heavy chain variable region of any one of SEQ ID NOS:6-8 and comprises the three CDRs of any one of SEQ ID NOS:6-8, a variant of at least one of the three CDRs in which 1 or 2 amino acids are substituted.

In some embodiments, a Siglec-7 binding domain of the present disclosure comprises a heavy chain variable region having at least 90%, 91%, 92%, 93%, 94%, 95%, 96%, 97%, 98%, 99%, or 100% sequence identity to the amino acid sequence of a heavy chain variable region of any one of SEQ ID NOS:6-8 and comprises the three CDRs of SEQ ID NO:6; or the three CDRs of SEQ ID NO:7, or the three CDRs of SEQ ID NO:8.

In some embodiments, a Siglec-7 binding domain of the present disclosure comprises a light chain variable region that comprises three CDRs of a variable region sequence as set forth SEQ ID NO:9 or 10, or a variant in which at least one of the three CDRs comprises 1, 2, 3, or 4 subsitutions. In some embodiments, a light chain variable region comprises three CDRs of a light chain variable region sequence as set forth in SEQ ID NO:9 or 10 in which at least one CDRs comprises 1 or two amino acid substitutions relative to the corresponding CDR of SEQ ID NO:9 or 10. In some embodiments, a light chain variable region comprises three CDRs of a variable region sequence as set forth in SEQ ID NO:9 or 10 in which at least one of the three CDRs has 1 amino acid substitution. In some embodiments, a light chain variable region comprises three CDRs of a variable region sequence as set forth in SEQ ID NO:9 or 10.

In some embodiments, a Siglec-7 binding domain of the present disclosure comprises a light chain variable region having at least 80%, 85%, 90%, 91%, 92%, 93%, 94%, 95%, 96%, 97%, 98%, or 99% sequence identity to the amino acid sequence of a light chain variable region of SEQ ID NO:9 or 10. In certain embodiments, a V_{L} sequence having at least 80%, 85%, 90%, 91%, 92%,93%,94%, 95%,96%, 97%,98%, or 99% identity to the amino acid sequence of a light chain variable region of SEQ ID NO:9 or 10 contains substitutions (e.g., conservative substitutions), insertions, or deletions relative to the reference sequence, but the binding domain retains the ability to bind to Siglec-7.

In some embodiments, a Siglec-7 binding domain of the present disclosure comprises a light chain variable region having at least 90%, 91%, 92%, 93%, 94%, 95%, 96%, 97%, 98%, or 99% sequence identity to the amino acid sequence of a light chain variable region of SEQ ID NO:9 or 10 and comprises the three CDRs of SEQ ID NO:9 or 10; or a variant of at least one of the three CDRs in which 1 or 2 amino acids are substituted.

In some embodiments, a Siglec-7 binding domain of the present disclosure comprises a light chain variable region having at least 90%, 91%, 92%, 93%, 94%, 95%, 96%, 97%, 98%, 99%, or 100% sequence identity to the amino acid sequence of a heavy chain variable region of SEQ ID NO:9 or 10 and comprises the three CDRs of SEQ ID NO:9; or the three CDRs of SEQ ID NO:10.

Fc engineering

In some embodiments, an anti-Siglec-9 antibody or bispecific or multispecific antibody of the present disclosure comprises an Fc region, which as described herein, may be a variant Fc region engineered to alter one or more functional properties of the antibody, such as extending serum half-life and/or reducing or enhancing effector function, including reducing complement fixation, Fc receptor binding, and/or antibody-dependent cell-mediated cytotoxicity. Furthermore, an antibody of the disclosure may be chemically modified (e.g., one or more chemical moieties can be attached to the antibody) or be modified to alter its glycosylation, again to alter one or more functional properties of the antibody. For purposes of describing amino acids present in an Fc region, the positions are numbered using EU index numbering where the designation “positon number X” means that X is an amino acid that is present at the indicated position. For purposes of describing mutations in an Fc region, the designation “X positon number Y” means that Y is an amino acid that is substituted for X relative to a reference sequence at the indicated position. For example, L234A, means that A is substituted for an L that occurs in a reference Fc region sequence at position 234. Similarly, the designation “-” when coupled with a position number, e.g., “position number-”, refers to a deletion, relative to a reference sequence, at the indicated position in the Fc region. For example, “236-” indicates that the residue at position 236 is deleted in an Fc region relative to a reference Fc region sequence.

In some embodiments, one or more amino acid modifications may be introduced into the Fc region of a bispecific or multispecific antibody, e.g., to increase stability and/or reduce or enhance binding of an Fc region to an Fc receptor. An Fc region variant may thus comprise a human Fc region sequence, such as a human IgG1, IgG2, IgG3, or IgG4 Fc region sequence, that comprises at least one amino acid modification, such as a substitution, compared to a native Fc region sequence. An Fc region variant may also comprise further modifications. Accordingly, in some embodiments, the Fc region variant has at least 80% identity, or at least 85%, at least 90%, or at least 95% identity to a native human IgG1, IgG2, IgG3, or IgG4 region and comprises specific Fc region modifications as described herein.

In some embodiments, an anti-Siglec-9 antibody or bispecific or multispecific antibody of the present disclosure having reduced effector function, e.g., reduced binding of the Fc region to an Fc receptor, includes one or more amino acid modifications; or two or more modifications. In some embodiments, at least one modification is at an Fc region position selected from the group consisting of positions 228, 233, 234, 235, 236, 237, 238, 268, 309, 322,327,330,331,233,242,259,287,292,297,302,306,323,332,334,252,254,256,232, 267, 328, and 329.

In some embodiments, an anti-Siglec-9 antibody or bispecific or multispecific antibody of the present disclosure that has reduced effector function, e.g., reduced binding of the Fc region to an Fc receptor, includes one or more amino acid modifications at an Fc region position selected from the group consisting of positions 234, 235, 236, 237, 238, 267, 268, 297, 309, 322, 327, 330, 331, and 328. In some embodiments, the antibody further comprises at least one modification in the Fc region at a position selected from the group consisting of positions 232, 233, 242, 252, 254, 256, 259, 287, 292, 302, 306, 323, 329, 332, and 334.

In some embodiments, an anti-Siglec-9 antibody or bispecific or multispecific antibody having Siglec-7 and Siglec-9 binding domains as described herein that has reduced effector function, e.g., reduced binding of the Fc region to an Fc receptor, comprises an Fc region having at least one residue selected from the group consisting of 228P, 233P, 234V, 234A/V, 234F, 235A, 235E, 235Q, 236-(i.e., a deletion of the residue at position 236), 237A, 2385, 242C, 252Y, 254T, 256E, 259C, 268A, 287C, 292C, 297C, 297A, 297G, 297Q, 302C, 306C, 309L, 322Q, 323C, 327G, 329G, 3305, 331S, 332C, and 334C. In some embodiments, an anti-Siglec-9 antibody as described herein that has reduced effector function, e.g., reduced binding of the Fc region to an Fc receptor, comprises an Fc region having at least two residues, or at least three residues, selected from the group consisting of 228P, 233P, 234V, 234A/V, 234F, 235A, 235E, 235Q, 236-(i.e., a deletion of the residue at position 236), 237A, 238S, 242C, 252Y, 254T, 256E, 259C, 268A, 287C, 292C, 297C, 297A, 297G, 297Q, 302C, 306C, 309L, 322Q, 323C, 327G, 329G, 3305, 331S, 332C, and 334C. In some embodiments, the antibody comprises an Fc region comprising 234A and 235A. In some embodiments, the antibody comprises an Fc region comprising 327G, 3305, and 331S. In some embodiments, the antibody comprises an Fc region comprising 327G, 330S, and 331 S. In some embodiments, the antibody comprises an Fc region comprising 233P, 234V, 235A, and 236-. In some embodiments, the antibody comprises an Fc region comprising 233P, 234V, and 235A. In some embodiments, the antibody comprises an Fc region comprising 233P, 234V, 235A, 236-, 327G, 330S, and 331S. In some embodiments, the antibody comprises an Fc region comprising 233P, 234V, 235A, 327G, 330S, and 331S. In some embodiments, the antibody comprises an Fc region comprising 297A/G/Q. In some embodiments, the antibody comprises an Fc region comprising 242C, 297C, and 334C. In some embodiments, the antibody comprises an Fc region comprising 287C, 297G, and 306C. In some embodiments, the antibody comprises an Fc region comprising 292C, 297G, and 302C. In some embodiments, the antibody comprises an Fc region comprising 297G, 323C, and 332C. In some embodiments, the antibody comprises an Fc region comprising 259C, 297G, and 306C. In some embodiments, the antibody comprises an Fc region comprising 234F, 235Q, 322Q, 252Y, 254T, and 256E. In some embodiments, the antibody comprises an Fc region comprising 234A, 235A, and 329G. In some embodiments, the antibody comprises an Fc region comprising 330S and 331S. In some embodiments, the antibody comprises an Fc region comprising 234A, 237A, 238S, 268A, 309L, 330S, and 33 1S. In some embodiments, the antibody comprises an Fc region comprising 233P, 234V, 235A, and 236-. In some embodiments, the antibody comprises an Fc region comprising 228P, 234V, and 23 5A. In some embodiments, the antibody comprises an Fc region comprising 228P and 235E/A.

In some embodiments, an anti-Siglec-9 antibody or bispecific or multispecific antibody of the present disclosure comprises an Fc region comprising a human IgG1 Fc region variant having decreased effector function, e.g., reduced binding of the Fc region to an Fc receptor. In some embodiments, the human IgG1 Fc region variant has at least 85%, identity to a native human IgG1 Fc region amino acid sequence. In some embodiments, the human IgG1 Fc region variant has at least 90% identity, or at least 95% identity, to a native human IgG1 Fc region amino acid sequence. In some embodiments, the Fc region comprises a human IgG1 Fc region variant having decreased effector function, e.g., reduced binding of the Fc region to an Fc receptor, and modifications to the Fc region that increase serum half-life of the antibody. In some embodiments, an anti-Siglec-9 antibody comprises a human IgG1 Fc region comprising amino acid modifications L234A and L235A. In some embodiments, an anti-Siglec-9 antibody comprises a human IgG1 Fc region comprising amino acid modifications A327G, A330S, and P331S. In some embodiments, an antibody comprises a human IgG1 Fc region comprising amino acid modifications E233P, L234V, L235A, which may further comprise an amino acid modification G236-, amino acid modifications G236-, A327G, A330S, and P331S; or amino acid modifications A327G, A330S, and P331S. In some embodiments, an anti-Siglec-9 antibody comprises a human IgG1 Fc region comprising an amino acid modification N297A/G/Q. In some embodiments, an antibody comprises a human IgG1 Fc region comprising amino acid modifications L242C, N297C, and K334C. In some embodiments, an antibody comprises a human IgG1 Fc region comprising amino acid modifications A287C, N297G, and L306C. In some embodiments, an antibody comprises a human IgG1 Fc region comprising amino acid modifications R292C, N297G, and V302C. In some embodiments, an antibody comprises a human IgG1 Fc region comprising amino acid modifications N297G, V323C, and I332C. In some embodiments, an antibody comprises a human IgG1 Fc region comprising amino acid modifications V259C, N297G, and L306C. In some embodiments, an antibody comprises a human IgG1 Fc region comprising amino acid modifications L234F, L235Q, K322Q, and M252Y, S254T, T256E. In some embodiments, an antibody comprises a human IgG1 Fc region comprising amino acid modifications L234A, L235A, and P329G. In some embodiments, an antibody comprises a human IgG1 Fc region comprising at least one amino acid modification, or at least two amino acid modifications selected from the group consisting of N297A, N297G, N297Q, N297C, D265A, L234A, L235A, G237A, C226S, C229S, E233P, L234V, L234F, L235E, L235Q, P329G, P331S, P331G, S267E, L328F, A287C,L306C,R292C, V259C, V302C, K322Q, V323 C, 1332C, A330L, A327G, A330S, L242C, and K334C.

In some embodiments, an anti-Siglec-9 antibody or bispecific or multispecific antibody of the present disclosure comprises an Fc region comprising a human IgG2 Fc region variant having decreased effector function, e.g., reduced binding of the Fc region to an Fc receptor. In some embodiments, the human IgG2 variant Fc region has at least 85%, identity to a native human IgG2 Fc region amino acid sequence. In some embodiments, the human IgG2 variant Fc region has at least 90% identity, or at least 95% identity to a native human IgG2 Fc region amino acid sequence. In some embodiments, the Fc region comprises a human IgG2 Fc region variant having decreased effector function, e.g., reduced binding of the Fc region to an Fc receptor, and modifications to the Fc region that increase serum half-life of the antibody. In some embodiments, an antibody comprises a human IgG2 Fc region comprising amino acid modifications A330S and P331S. In some embodiments, an antibody comprises a human IgG2 Fc region comprising amino acid modifications V234A, G237A, P238S, H268A, V309L, A330S, and P331S. In some embodiments, an antibody comprises a human IgG2 Fc region comprising at least one amino acid modification V234A, G237A, H268Q, V309L, A330S, P331S, C232S, C233 S, S267E, L328F, M252Y, S254T, and T256E. In some embodiments, an antibody comprises a human IgG2 Fc region comprising one or more modifications, or two or more modifications at a position selected from the group consisting of V234A, G237A, P238S, H268Q, V309L, A330S, P331S S267E, S267E, and L328F.

In some embodiments, an anti-Siglec-9 antibody or bispecific or multispecific antibody of the present disclosure comprises an Fc region comprising a human IgG4 Fc region variant having decreased effector function, e.g., reduced binding of the Fc region to an Fc receptor. In some embodiments, the human IgG4 variant Fc region has at least 85%, identity to a native human IgG4 Fc region amino acid sequence. In some embodiments, the human IgG4 variant Fc region has at least 90% identity, or at least 95% identity to a native human IgG4 Fc region amino acid sequence. In some embodiments, the Fc region comprises a human IgG4 Fc region variant having decreased effector function, e.g., reduced binding of the Fc region to an Fc receptor, and modifications to the Fc region that increase serum half-life of the antibody. In some embodiments, an antibody comprises a human IgG4 Fc region comprising amino acid modifications E233P, F234V, L235A, and G236-. In some embodiments, an antibody comprises a human IgG4 Fc region comprising amino acid modifications E233P, F234V, and L235A. In some embodiments, an antibody comprises a human IgG4 Fc region comprising amino acid modifications S228P and L235E/A. In some embodiments, an antibody comprises a human IgG4 Fc region comprising at least one modification selected from the group consisting of E233P, L235A or L235E, G237A, L236E, S267E, E318A, and L328F.

In certain embodiments, the proline at position 329 of a wild-type human Fc region of a bispecific or multispecific antibody of the present disclosure is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcγRIII (Sondermann et al.: Nature 406, 267-273 (20 Jul. 2000)). In certain embodiments, the antibody comprises at least one further amino acid substitution. In one embodiment, the further amino acid substitution is S228P, E233P, L234A, L235A, L235E, N297A, N297D, or P331 S. In some embodiments, the at least one further amino acid substitution is L234A and L235A of the human IgG1 Fc region or S228P and L235E of the human IgG4 Fc region (see e.g., US 2012/0251531). In another embodiments, the at least one further amino acid substitution is L234A and L235A and P329G of the human IgG1 Fc region.

In some embodiments, an anti-Siglec-9 antibody or bispecific or multispecific antibody of the present disclosure having reduced effector function, e.g., reduced binding of the Fc region to an Fc receptor, includes one more amino acid substitutions at an Fc region positions selected from the group consisting of 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In some embodiments, an anti-Siglec-9 antibody or bispecific or multispecific antibody having reduced effector function, e.g., reduced binding of the Fc region to an Fc receptor, comprises CH domains from different human IgG isotypes. For examples, in some embodiments, an antibody may comprise an IgG2 CH1 domain and hinge sequence, such as the sequence ASTKGPSVFPLAPCSRSTSESTAALGCLVKDYFPEPVTVSWNSGALTSGVHTFPAVLQS SGLYSLSSVVTVPSSNFGTQTYTCNVDHKPSNTKVDKTVERKCCVECPPCP (SEQ ID NO:24), and an IgG1 CH2 and CH3 domain sequence that comprises an amino acid substitution S267E and/or L328F; and/or an N297A or N297Q substitution. In some embodiments, an antibody having reduced effector function comprises human IgG2 and IgG4 sequences. For example, such an antibody may comprise amino acids 117 to 260 of human IgG2 and amino acids 261 to 447 of human IgG4.

In some embodiments, an agonist antibody of the present disclosure exhibits enhanced Fc receptor binding and/or cross-linking activity. In some embodiments, such an antibody comprises an Fc region comprising one or more amino acid modifications; or two or more modifications. In some embodiments, at least one modification is at an Fc region position selected from the group consisting of positions 243, 292, 300, 305, 396, 239, 332, 298, 333,334,234,235,236,239,268,270,298,270,326,330,334,267,324,345,430,and 440.

In some embodiments, such an antibody comprises at least one of the following combinations of mutations, as designated with respect to human IgG1 subclass: F243L/R292P/Y300L/V305I/P396L; S239D/I332E; S239D/I332E/A330L; S298A/E333A/K334A; in one heavy chain L234Y/L235Q/G236W/S239M/H268D/D270E/S298A and in a heavy chain with which it dimerizes, D270E/K326D/A330M/K334E; G236A/S239D/I332E; K326W/E333S; S267E/H268F/S324T; and E345R/E430G/S440Y. In some embodiments, an antibody having enhanced FcR binding; and/or cross-linking activity comprises an IgG1/IgG3 cross subclass (see, e.g., Natsume eta/, Can Res 68:3863-3872, 2008).

In some embodiments, an agonist antibody may comprise one or more mutations in the Fc region that increases binding to FcγRIIb.

In some embodiments, an agonist antibody comprises mutations that increase half-life, e.g., M252Y/S254T/T256E or M428L/N434S (relative to human IgG1 sublcass).

Effector function or antibody half-life can be evaluated using any suitable assay. For example, in vitro and/or in vivo cytotoxicity assays can be conducted to confirm reduced CDC and/or ADCC activities. In some embodiments, Fc receptor binding assays can be conducted to determine binding to an FcγR. Non-limiting examples of in vitro assays to assess ADCC activity are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82: 1499-1502 (1985); 5,821,337 (see Bruggemann, M. et al., J. Exp. Med. 166: 1351-1361 (1987)). Alternatively, non-radioactive assays methods may be employed (see, for example, ACTI™ non-radioactive cytotoxicity assay for flow cytometry (CellTechnology, Inc.

Mountain View, CA; and CytoTox 96® non-radioactive cytotoxicity assay (Promega, Madison, WI).

Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to assess binding to C1q and CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)).

In some embodiments, an antibody comprising a variant Fc region that has reduced effector function, reduced binding of the Fc region to an Fc receptor, and/or reduces cross-linking of Siglec receptors, has at least a 30%, or 50% or greater, reduction in binding, effector function, or cross-linking compared to the widltype counterpart Fc region when the variant is compared to the wildtype using the same assay and antibody format.

In some embodiments, an antibody comprising a variant Fc region that has enhanced effector function, increased binding of the Fc region to an Fc receptor, and/or increased cross-linking of Siglec receptors, has at least a 30%, or 50% or greater, increase in binding, effector function, or cross-linking compared to the widltype counterpart Fc region when the variant is compared to the wildtype using the same assay and antibody format.

An anti-Siglec-9 antibody or bispecific or multispecific antibodies of the present disclosure comprising an Fc region having increased or decreased effector function, e.g., reduced or enhanced binding to an Fc receptor compared to the counterpart native Fc region sequence, may also comprises modification to increase serum half-life. In some embodiments, FcRn binding ability, which typically correlates with serum half-life may also be evaluated. Antibodies with increased half-lives and improved binding to the neonatal Fc receptor (FcRn) are described in US2005/0014934A1 (Hinton et al.). Those antibodies comprise an Fc region with one or more substitutions therein which improve binding of the Fc region to FcRn. Such Fc variants include those with substitutions at one or more of Fc region residues:238,256,265,272,286,303,305,307,31 1,312,317,340,356,360,362,376,378, 380, 382, 413, 424 or 434, e.g., substitution of Fc region residue 434 (U.S. Pat. No. 7,371,,826). See also Duncan & Winter, Nature 322:738-40 (1988); U.S. Pat. Nos. 5,648,260; 5,624,821; and WO 94/29351 concerning other examples of Fc region variants. Additional mutations that improve serum half-life include YTE mutations, i.e., amino acids Y, T, and E at positions 252, 254, and 256, respectively. FcRn binding and in vivo clearance/half-life determinations can be performed using methods known in the art (see, e.g., Petkova, S. B. et al., Int'l. Immunol. 18(12): 1759-1769 (2006)). In typical embodiments, a variant Fc region that confers increased stability, increases serum half-life by at least 5%, or at least 10%, at least 20%, or greater, when the variant is compared to the counterpart wild-type antibody.

In some embodiments, biological half-life can be increased by modifying the Fc region within the CH1 or CL region to contain a salvage receptor binding epitope taken from two loops of a CH2 region of an Fc region of an IgG, as described in U.S. Pat. Nos. 5,869,046 and 6,121,022 by Presta et al. In some embodiments, stabilizing mutations, e.g., at cysteine positions C232 and C233 of human IgG2, are introduced to prevent disulfide bond exchange and stabilize the human IgG2 in the IgG2-A conformation.

In still another embodiment, the glycosylation of an antibody is modified. For example, an aglycoslated antibody or antibody having an altered glycosylation pattern can be made. Glycosylation can be altered, for example, to increase the affinity of the antibody for an antigen or, if made in the Fc region, to influence effector function. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

### Methods of Producing Bispecific or Multispecific Antibodies

Antibodies can be produced using any number of expression systems, including prokaryotic and eukaryotic expression systems (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. In some embodiments, the expression system is a mammalian cell expression, such as a CHO cell expression system. Additional illustrative mammalian cell lines for production of antibody heavy and light chains include, but are not limited to, CV1, BHK, TM4, VERO, MDCK, and HepG2 cells.

A host cell transfected with one or more expression vectors encoding an antibody heavy and/or light chain of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

### Treatment of Cancer

Neutralizing anti-Siglec-9 antibodies as described herein, and/or neutralizing bispecific or multispecific antibodies comprising a Siglec-9 and Siglec-7 binding domain of the present disclosure can be used to inhibit tumor growth.

Any cancer can be treated with a neutralizing anti-Siglec-9 antibody, or a neutralizing bispecific or a multispecific antibody, of the present disclosure. In some embodiments, the cancer is a carcinoma or a sarcoma. In some embodiments, the cancer is a hematological cancer. In some embodiments, the cancer is breast cancer, prostate cancer, testicular cancer, renal cell cancer, bladder cancer, ovarian cancer, cervical cancer, endometrial cancer, lung cancer, colorectal cancer, anal cancer, pancreatic cancer, gastric cancer, esophageal cancer, hepatocellular cancer, head and neck cancer, glioblastoma, mesothelioma, melanoma, or a bone or soft tissue sarcoma. In some embodiments, the cancer is acute lymphoblastic leukemia, acute myeloid leukemia, adrenocortical carcinoma, anal cancer, appendix cancer, astrocytoma, basal-cell carcinoma, bile duct cancer, bladder cancer, bone tumor, brainstem glioma, brain cancer, cerebellar astrocytoma, cerebral astrocytoma, ependymoma, medulloblastoma, supratentorial primitive neuroectodermal tumors, visual pathway and hypothalamic glioma, breast cancer, bronchial adenomas, Burkitt's lymphoma, central nervous system lymphoma, cerebellar astrocytoma, cervical cancer, chondrosarcoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, epitheliod hemangioendothelioma (EHE), esophageal cancer, Ewing's sarcoma, extracranial germ cell tumor, extragonadal germ cell tumor, extrahepatic bile duct cancer, eye cancer, intraocular melanoma, retinoblastoma, gallbladder cancer, gastric cancer, gastrointestinal carcinoid tumor, gastrointestinal stromal tumor (GIST), germ cell tumor, gestational trophoblastic tumor, gastric carcinoid, hairy cell leukemia, head and neck cancer, heart cancer, hepatocellular cancer, Hodgkin lymphoma, hypopharyngeal cancer, hypothalamic and visual pathway glioma, childhood, intraocular melanoma, islet cell carcinoma, Kaposi sarcoma, kidney cancer, laryngeal cancer, leukaemias, lip and oral cavity cancer, liposarcoma, liver cancer, non-small cell lung cancer, small-cell lung cancer, lymphomas, macroglobulinemia, male breast cancer, malignant fibrous histiocytoma of bone, medulloblastoma, melanoma, Merkel cell cancer, mesothelioma, metastatic squamous neck cancer, mouth cancer, multiple endocrine neoplasia syndrome, multiple myeloma, mycosis fungoides, myelodysplastic syndromes, myelogenous leukemia, myeloid leukemia, adult acute, myeloproliferative disorders, chronic, myxoma, nasal cavity and paranasal sinus cancer, nasopharyngeal carcinoma, neuroblastoma, non-Hodgkin lymphoma, oligodendroglioma, oral cancer, oropharyngeal cancer, osteosarcoma, ovarian cancer, ovarian epithelial cancer, ovarian germ cell tumor, ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma, supratentorial primitive neuroectodermal tumors, pituitary adenoma. plasma cell neoplasia, pleuropulmonary blastoma, primary central nervous system lymphoma, prostate cancer, rectal cancer, renal cell carcinoma, retinoblastoma, rhabdomyosarcoma, salivary gland cancer, Ewing sarcoma, Kaposi sarcoma, soft tissue sarcoma, uterine sarcoma, Sezary syndrome, non-melanoma skin cancer, melanoma Merkel cell skin carcinoma, small intestine cancer, squamous cell carcinoma, squamous neck cancer, stomach cancer, cutaneous T-Cell lymphoma, testicular cancer, throat cancer, thymoma, thyroid cancer, transitional cell cancer of the renal pelvis and ureter, trophoblastic tumor, gestational, urethral cancer, uterine cancer, vaginal cancer, vulvar cancer, Waldenstrom macroglobulinemia, or Wilms tumor.

A neutralizing anti-Siglec-9 antibody or neutralizing bispecific or multispecific antibody may be administered with one or more additional therapeutic agents, e.g., chemotherapeutic agents and/or additional immunotherapies.

In some embodiments, a neutralizing anti-Siglec-9 antibody or neutralizing bispecific or multispecific antibody can be administered in conjunction with another checkpoint inhibitor. In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In certain embodiments, the checkpoint inhibitor inhibits a checkpoint protein which may be CTLA-4, PDL1, ICOS, PDL2, IDO1, ID02, PDl, B7-H3, B7-H4, BTLA, HVEM, TIM3, GAL9, GITR, HAVCR2, LAG3, KIR, LAIR1, LIGHT, MARCO, OX-40, SLAM,, 2B4, CD2, CD27, CD28, CD30, CD40, CD70, CD80, CD86, CD137, CD160, CD39, VISTA, TIGIT, CGEN-15049, 2B4, CHK 1, CHK2, A2aR, B-7 family ligands or a combination thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is ICOS.

In some embodiments, a neutralizing anti-Siglect 9 antibody or neutralizing bispecific or multispecific antibody can be administered in conjunction with a therapeutic antibody, such as an antibody that targets a tumor cell antigen. Examples of therapeutic antibodies include as rituximab, trastuzumab, tositumomab, ibritumomab, alemtuzumab, epratuzumab, bevacizumab, elotuzumab, necitumumab, blinatumomab, brentuximab, cetuximab, daratumumab, denosumab, dinutuximab, gemtuzumab ibritumomab ipilimumab, nivolumab, obinutuzumab, ofatumumab, ado-trastuzumab, panitumumab, pembrolizumab, pertuzumab, ramucirumab, and ranibizumab.

In some embodiments, a neutralizing anti-Siglec-9 antibody or neutralizing bispecific or multispecific antibody is administered with a chemotherapeutic agent. Examples of cancer chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as bexarotene, alitretinoin; denileukin diftitox; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, mifepristone, aromatase inhibiting 4(5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, crizotinib, dasatinib, erlotinib, fostamatinib, gefitinib, imatinib, lapatinib, lenvatinib, mubritinib, nilotinib, pazopanib, pegaptanib, ruxolitinib, vandetanib, vemurafenib, and sunitinib; sirolimus (rapamycin), everolimus and other mTOR inhibitors. Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Illustrative chemotherapeutic agents additionally include paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinasel and other inhibitors of proteins or enzymes known to be upregulated, over-expressed or activated in cancers, the inhibition of which down regulates cell replication. Additional agents include asparaginase and a Bacillus Calmete-Guerin preparation.

A neutralizing anti-Siglec-9 antibody or neutralizing bispecific or multispecific antibody may also be administered to a cancer patient in conjunction with a cell based therapy, such as NK cell therapy or a cancer vaccine. In some instances, a cancer vaccine is a peptide-based vaccine, a nucleic acid based vaccine, a cell-based vaccine, a virus-based or viral fragment based vaccine or an antigen presenting cell (APC) based vaccine (e.g. dendritic cell based vaccine). Cancervaccines include Gardasil®, Cervarix®, sipuleucel-T (Provenge®), NeuVax™, HER-2 ICD peptide-based vaccine, HER-2/neu peptide vaccine, AdHER2/neu dendritic cell vaccine, HER-2 pulsed DC1 vaccine, Ad-sig-hMUC-1/ecdCD40L fusion protein vaccine, MVX-ONCO-1, hTERT/survivin/CMV multipeptide vaccine, E39, J65, PlOs-PADRE, rV-CEA-Tricom, GVAX®, Lucanix®, HER2 VRP, AVX901, ONT-10, ISAlOl, ADXS1 1-001, VGX-3 100, INO-9012, GSK1437173A,BPX-501, AGS-003, IDC-G305, HyperAcute®-Renal (HAR) immunotherapy, Prevenarl3, MAGER-3.A1, NA17.A2, DCVax-Direct, latent membrane protein-2 (LMP2)-loaded dendritic cell vaccine (NCT02115126), HS410⁻¹⁰¹ (NCT02010203, Heat Biologies), EAU RF 2010⁻⁰¹ (NCT01435356, GSK), 140036 (NCT02015104, Rutgers Cancer Institute ofNew Jersey), 130016 (NCTO 1730118, National Cancer Institute), MVX-201101 (NCT02193503, Maxivax SA), ITL-007-ATCR-MBC (NCT01741038, Immunovative Therapies, Limited), CDR0000644921 (NCT00923143, Abramson cancer center of the University of Pennsylvania), SuMo-Sec-01 (NCT00108875, Julius Maximilians UniversitaetHospital), or MCC-15651 (NCT01176474, Medarex, Inc, BMS).

### Treatment of Inflammatory and Autoimmune Disease

In some embodiments, an agonist anti-Siglec-9 antibody as described herein or an agonist bispecific or multispecific antibody of the present disclosure is used to treat an immune disorder, such as an inflammatory disease or autoimmune disease, or conditions in which it is desirable to suppress an immune response, e.g., organ transplant. In such embodiments, an agonist anti-Siglec-9 antibody or agonist bispecific or multispecific antibody typically comprises an Fc region, e.g., a human IgG1 or active variant thereof.

Representative inflammatory or autoimmune disorders that can be treated with an agonist bispecific or multispecific antibody include, but are not limited to rheumatoid arthritis, systemic lupus erythematosus, multiple sclerosis, a spondyloarthropathy, such as ankylosing spondylitis, autoimmune Addison's disease, an antibody-mediated inflammatory or autoimmune disease, graft versus host disease, sepsis, psoriasis, psoriatic arthritis, atherosclerosis, Sjogren's syndrome, progressive systemic sclerosis, scleroderma, acute coronary syndrome, ischemic reperfusion injury, Crohn's Disease, acute and chronic colitis, ulcerative colitis, inflammatory bowel disease, glomerulonephritis, IgA nephropathy, polycystic kidney disease, myasthenia gravis, idiopathic pulmonary fibrosis, fibrotic disease (e.g., pulmonary fibrosis, liver cirrhosis, atrial fibrosis, endomyocardial fibrosis, myelofibrosis, or retroperitoneal fibrosis), chronic obstructive pulmonary disease, asthma, atopic dermatitis, acute respiratory distress syndrome (ARDS), vasculitis, Raynaud's disease and the like.

In some embodiment, the inflammatory disorder is the result of infection with a pathogen, e.g., viral or bacterial pathogen.

In some embodiments, an agonist anti-Siglec-9 antibody or agonist bispecific or multispecific antibody as described herein is administered to prevent or treat transplant rejection.

In some embodiments, the antibody may be administered in conjunction with an additional therapy to treat an inflammatory or autoimmune disease, or for transplant rejection. Additional therapeutic agents include, but are not limited to, hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective antiinflammatory peptides (ImSAIDs) such as phenylalanine-glutamine-glycine (FEG) and its D-isomeric form (feG) (IMULAN BioTherapeutics, LLC); anti-rheumatic drugs such as azathioprine, cyclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMERA®); interleukin 13 (IL-13) blockers such as lebrikizumab; interferon alpha (IFN) blockers such as rontalizumab; Beta 7 integrin blockers such as rhuMAb Beta7; IgE pathway blockers such as anti-Mi prime; secreted homotrimeric LTa3 and membrane bound heterotrimer LTa1/p2 blockers such as Anti-lymphotoxin alpha (LTa), autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate, (DIDROCAL®), NE-58095, zoledronic acid/zoledronate (ZOMETA®), alendronate (FOSAMAX®), pamidronate (AREDIA®), tiludronate(SKELID®), orrisedronate (ACTONEL®); COX-2 inhibitor (e.g. celecoxib or etoricoxib); and non-steroidal anti-inflammatory drugs with analgesic, antipyretic and anti-inflammatory effects. NSAIDs include non-selective inhibitors of the enzyme cyclooxygenase. Specific examples ofNSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid. Additional agents include an agonistic anti-CTLA-4 agent, an agonistic anti-PD-1 agent, an agonistic anti-PD-L1 agent, an agonistic anti-PD-L2 agent, an agonistic anti-CD27 agent, an agonistic anti-CD30 agent, an agonistic anti-CD40 agent, an agonistic anti-4-1 BB agent, an agonistic anti-GITR agent, an agonistic anti-OX40 agent, an agonistic anti-TRAILR1 agent, an agonistic anti-TRAILR2 agent, an agonistic anti-TWEAK agent, an agonistic anti-TWEAKR agent, an agonistic anti-cell surface lymphocyte protein agent, an agonistic anti-BRAF agent, an agonistic anti-MEK agent, an agonistic anti-CD33 agent, an agonistic anti-CD20 agent, an agonistic anti-HLA-DR agent, an agonistic anti-FI LA class I agent, an agonistic anti-CD52 agent, an agonistic anti-A33 agent, an agonistic anti-GD3 agent, an agonistic anti-PSMA agent, an agonistic anti-Ceacan 1 agent, an agonistic anti-Galedin 9 agent, an agonistic anti-HVEM agent, an agonistic anti-VISTA agent, an agonistic anti-B7 H4 agent, an agonistic anti-HHLA2 agent, an agonistic anti-CD155 agent, an agonistic anti-CD80 agent, an agonistic anti-BTLA agent, an agonistic anti-CD160 agent, an agonistic anti-CD28 agent, an agonistic anti-CD226 agent, an agonistic anti-CEACAMI agent, an agonistic anti-TIM3 agent, an agonistic anti-TIGIT agent, an agonistic anti-CD96 agent, an agonistic anti-CD70 agent, an agonistic anti-CD27 agent, an agonistic anti-LIGHT agent, an agonistic anti-CD137 agent, an agonistic anti-DR4 agent, an agonistic anti-CR5 agent, an agonistic anti-TNFRS agent, an agonistic anti-TNFR1 agent, an agonistic anti-FAS agent, an agonistic anti-CD95 agent, an agonistic anti-TRAIL agent, an agonistic anti-DR6 agent, an agonistic anti-EDAR agent, an agonistic anti-NGFR agent, an agonistic anti-OPG agent, an agonistic anti-RANKL agent, an agonistic anti-LTP receptor agent, an agonistic anti-BCMA agent, an agonistic anti-TACI agent, an agonistic anti-BAFFR agent, an agonistic anti-EDAR2 agent, an agonistic anti-TROY agent, and an agonistic anti-RELT agent. For example, the agent may be an agonistic anti-CTLA-4 agent, an agonistic anti-PD-1 agent, or an agonistic anti-PD-L1 agent.

### Administration of Antibodies

An atti-Siglec-9 antibody or bispecific or multispecific antibody of the present disclosure is administered to a patient in a therapeutically effective amount using a dosing regimen suitable for treatment of disease or disorder, e.g., cancer or an inflammatory disease/autoimmune disorder as described above. The composition can be formulated for use in a variety of drug delivery systems. One or more physiologically acceptable excipients or carriers can also be included in the compositions for proper formulation. Suitable formulations for use in the present disclosure are found, e.g., in Remington: The Science and Practice of Pharmacy, 21st Edition, Philadelphia, PA. Lippincott Williams & Wilkins, 2005.

The anti-Siglec-9 antibody or bispecific or multispecific antibody is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxillary pharmaceutical substances as required to approximate physiological conditions, such as pH adjusting and buffering agents, tonicity adjusting agents, and the like.

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include amelioration of symptoms of the disease in the patient. Amounts effective for this use will depend upon the severity of the disease and the general state of the patient's health, including other factors such as age, weight, gender, administration route, etc. Single or multiple administrations of the antibody may be administered depending on the dosage and frequency as required and tolerated by the patient. In any event, the methods provide a sufficient quantity of antibody to effectively treat the patient.

The antibody can be administered by any suitable means, including, for example, parenteral, intrapulmonary, and intranasal, administration, as well as local administration, such as intratumor administration. Parenteral infusions include intramuscular, intravenous, intraarterial, intraperitoneal, or subcutaneous administration. In some embodiments, the antibody may be administered by insufflation. In an illustrative embodiment, the antibody may be stored at 10 mg/ml in sterile isotonic aqueous saline solution for injection at 4° C. and is diluted in either 100 ml or 200 ml 0.9% sodium chloride for injection prior to administration to the patient. In some embodiments, the antibody is administered by intravenous infusion over the course of 1 hour at a dose of between 0.01 and 25 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.01 mg/kg body weight to about 25 mg/kg body weight or in 10 the range 1 mg-2 g per patient. Preferably the dose is in the range 0.1-10 mg/kg or approximately 50 mg-1000 mg/patient. The dose may be repeated at an appropriate frequency which may be in the range once per day to once every three months, or every six months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the antibody is administered approximately once per month.

### OTHER EMBODIMENTS

In additional embodiments, the disclosure provides a neutralizing bivalent or multispecific antibody comprising an anti-Siglec-7 and anti-Siglec-9 antibody as described herein for use in a method of inhibiting cancer cell proliferation in a subject that has cancer, e.g., in a tumor that comprises tumro cells and/or immune cells that express Siglec-9 and Siglec-7 ligands, for use in the preparation of a medicament for inhibiting cancer cell proliferation.

In other embodiments, the disclosure provides an agonist bivalent or multispecific antibody comprising an anti-Siglec-7 and anti-Siglec-9 antibody as described herein for use in a method of inhibiting an immune response in a subject or for use in the preparation of a medicament for inhibiting an immune response. In some embodiments, the subject has an autoimmune disorder.

The following examples are offered for illustrative purposes, and are not intended to limit the invention. Those of skill in the art will readily recognize a variety of non-critical parameters that can be changed or modified to yield essentially the same results.

### EXAMPLES

### Example 1. Siglec-9 Ligand Detection on Tumor Cells and Immune Cells

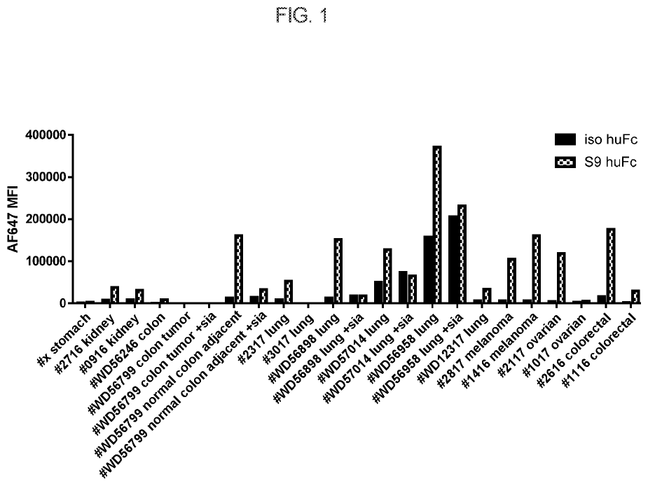

Ligand levels on primary tumor cells were evaluated (FIG. 1). Specific binding of recombinant Siglec-9-Fc fusion protein was used to assess Siglec-9 ligand levels on cells isolated form fresh b primary human tumors. As a specificity control, cells were treated with sialidase/neuraminidase (Roche) at 0.1U/mL to remove sialic acids from the cell surface. The results demonstrated that Siglec-9 ligands are detected on tumor cells from various subsets of tumors including from colon, lung, ovarian, melanoma and kidney, at different levels.

### Example 2. Siglec-9 Detection on Tumor-Infiltrating T Cells

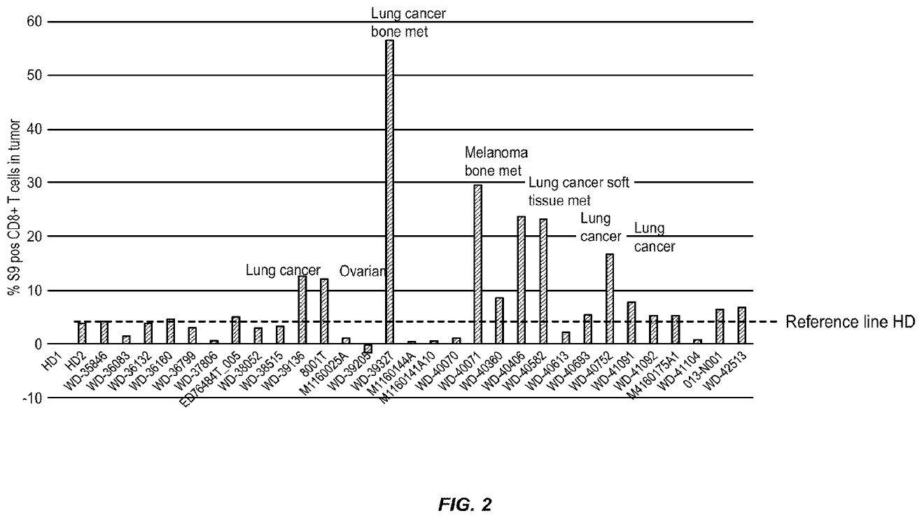

Samples comprising cells from primary human tumor specimens were prepared using the Mylteni GentleMACS instrument according to the manufacturer's instructions. Cells were analyzed by fluorescent-activated cell sorting to determine immune cell surface markers including CD3, CD8, CD16, CD45 and 7-AAD as a viability marker. CD8+ T cells were identified and gated as 7-AAD-CD45+CD3+CD8+. Anti-Siglec-9-PE using anti-Siglec-9 monoclonal antibody MAB1139 (R&D Systems) was used to detect Siglec-9 levels. The results demonstrated that the percentage of Siglec-9-expressing CD8+tumor-infiltrating T cells is enhanced in a subset of tumors (FIG. 2).

### Example 3. Antibodies with Siglec-9 Ligand Blocking Activity

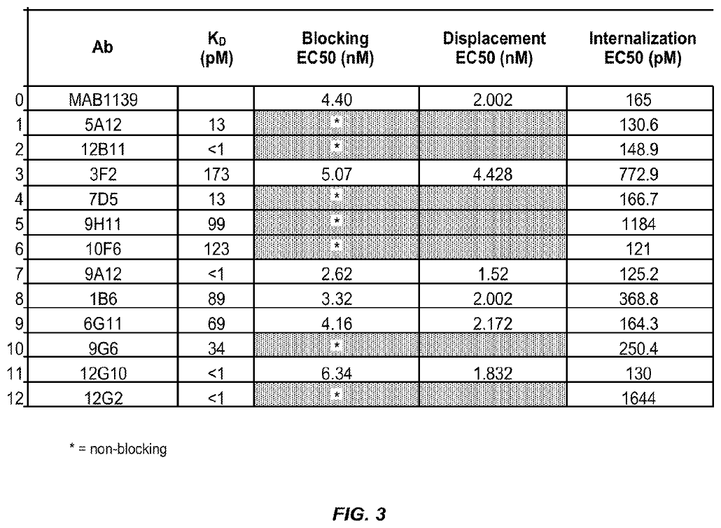

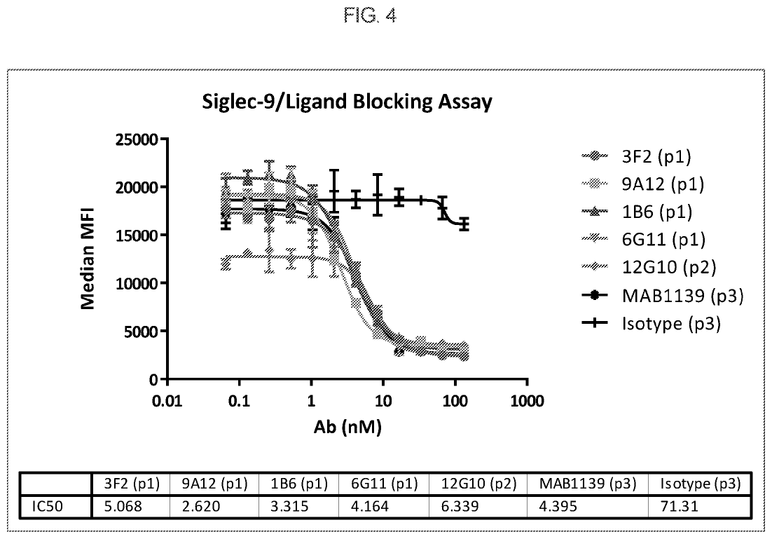

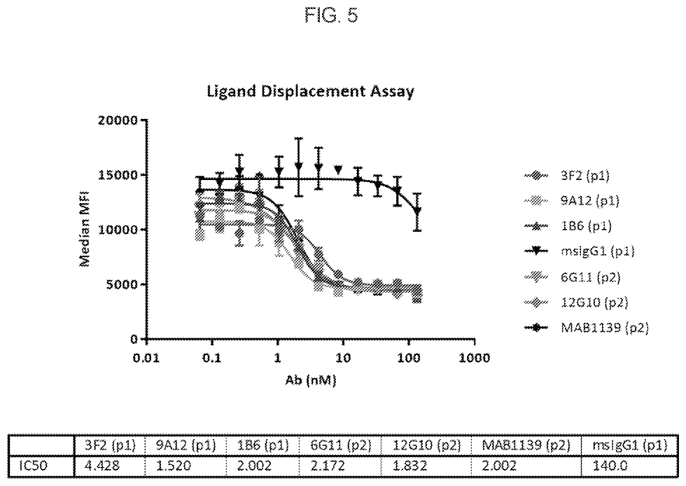

Recombinant Siglec-9-Fc was added to T47D cells in the presence of anti-Siglec-9 antibodies at increasing concentrations and binding of the complex was detected on the cell surface. The results showed that anti-Siglec-9 antibodies blocked the interaction of Siglec-9 with ligands present on the surface of T47D breast cancer cells with various potencies (FIGS. 3 and 4). The ability of an antibody to displace ligand binding was also determined (FIGS. 3 and 5).

### Example 4. Antibodies with Siglec-9 Internalization Activity

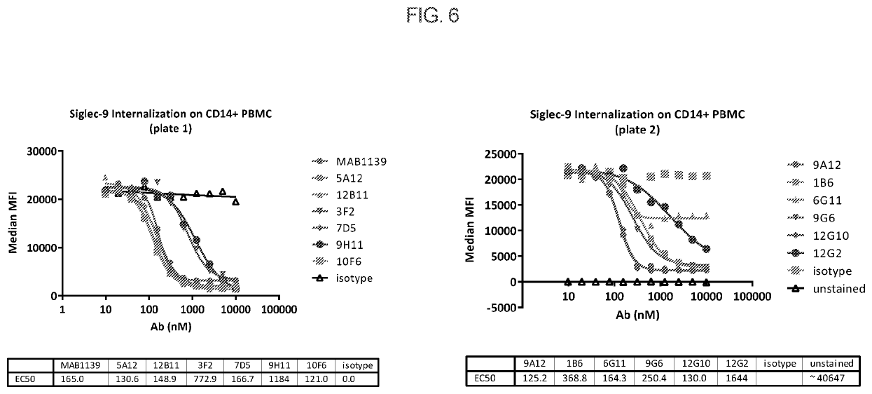

Primary human peripheral blood mononuclear cells (PBMC) were incubated with increasing concentrations of anti-Siglec-9 antibodies for 24 hours and remaining Siglec-9 in the cell surface of monocytes was detected using a non-competing anti-Siglec-9 antibody. The results showed that anti-Siglec-9 antibodies caused internalization of Siglec-9 on primary human monocyte cells with various potencies. (FIGS. 3 and 6).

### Example 5. Effect of Anti-S9 Antibody on Dendritic Cell Differentiation

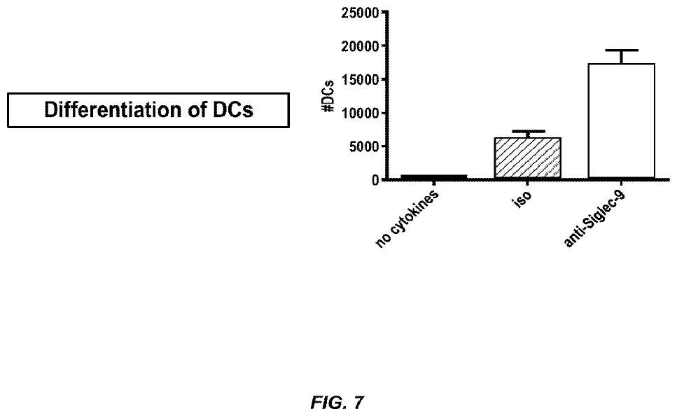

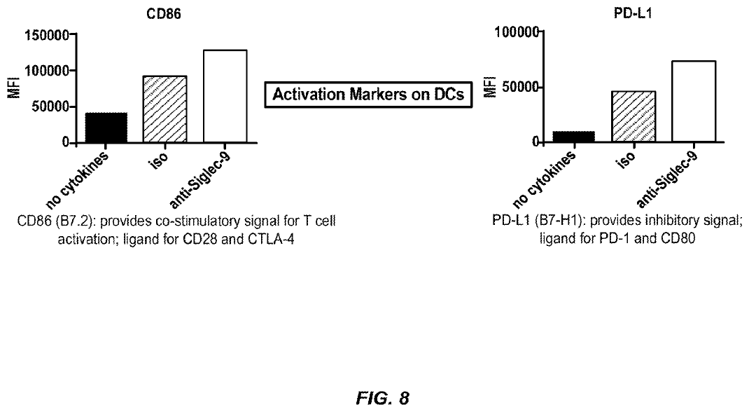

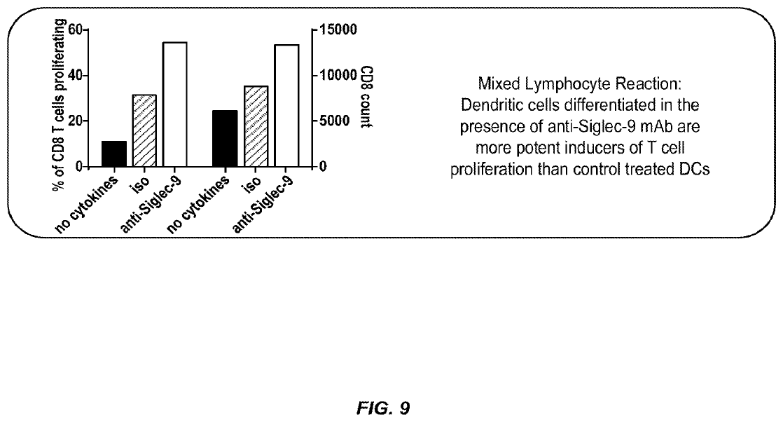

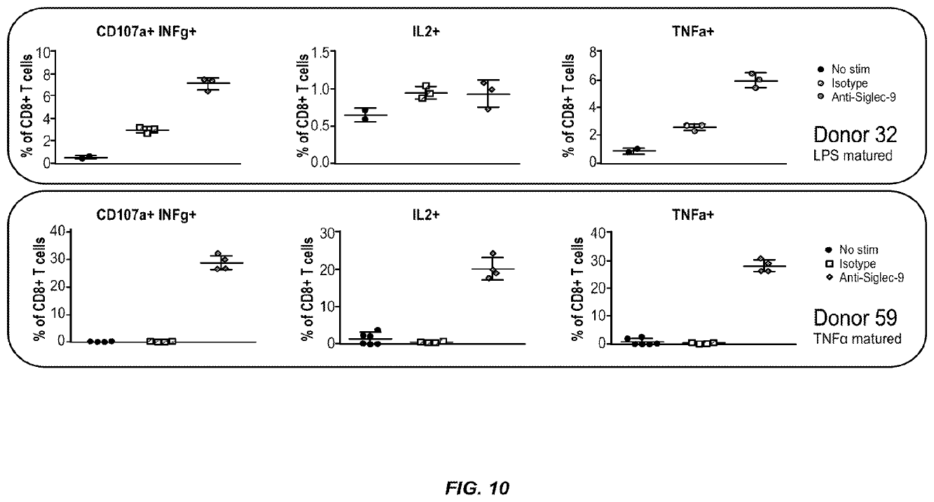

Differentiation of primary monocytes from healthy donors into dendritic cells in the presence of anti-Siglec-9 antibody leads to increased numbers of mature cells (FIG. 7, higher levels of activation markers (FIG. 8) and increased potential to stimulate proliferation in mixed lymphocyte reactions (FIG. 9) and in autologous peptide presentation and T cell activation assays. (FIG. 10).

### Example 6. Effect of Anti-S9 Antibody on Macrophage Cytokine Secretion

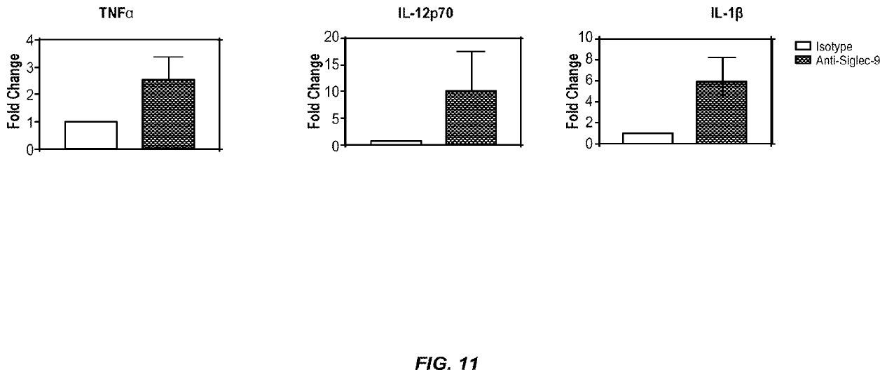

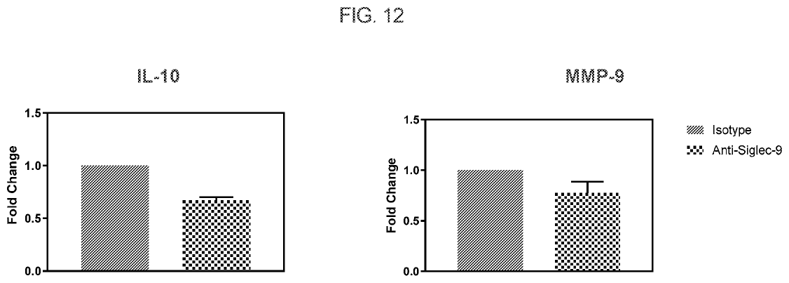

Differentiation of primary monocytes from healthy donors into macrophages in the presence of anti-Siglec-9 antibody led to increased production of inflammatory cytokines by M1 type macrophages (FIG. 11), and reduced production of immune-suppressive cytokines from M2 type macrophages (FIG. 12)

### Example 7. Agonist Anti-Siglec-9 Antibody Inhibits the Production of Pro-Inflammatory Cytokines in CD16+Monocytes Stimulated by LPS

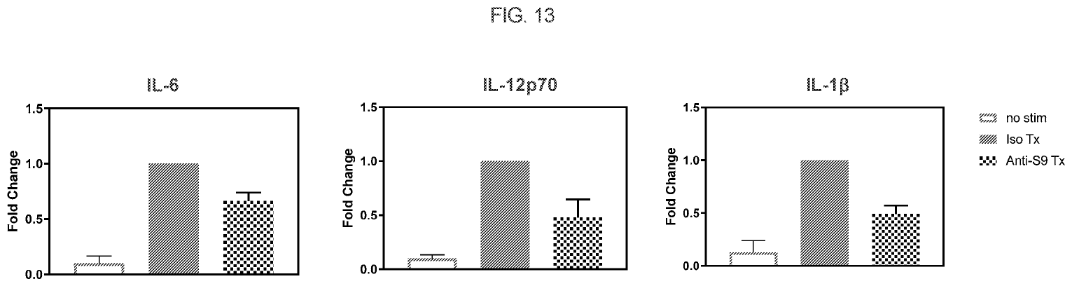

Monocytes were prepared as described in the Methods section. LPS stimulation of CD16+ monocytes lead to production of inflammatory cytokines, which was inhibited in the presence of an agonistic (FcR engaging) anti-Siglec-9 antibody (FIG. 13).

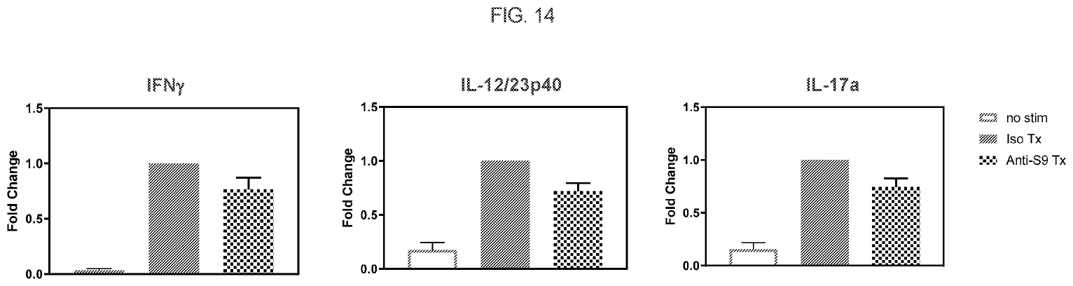

FIG. 14 provides data illustrating inhibition of the production of proinflammatory cytokines from an autologous co-culture of CD16+ monocytes with T cells after T cell stimulation with CD3/CD28 in the presence of an agonistic anti-Siglec-9 antibody.

### Example 8. Antibody 9A12 Agonistic Activity an Acute Bleomycin-Induced Lung Fibrosis Model

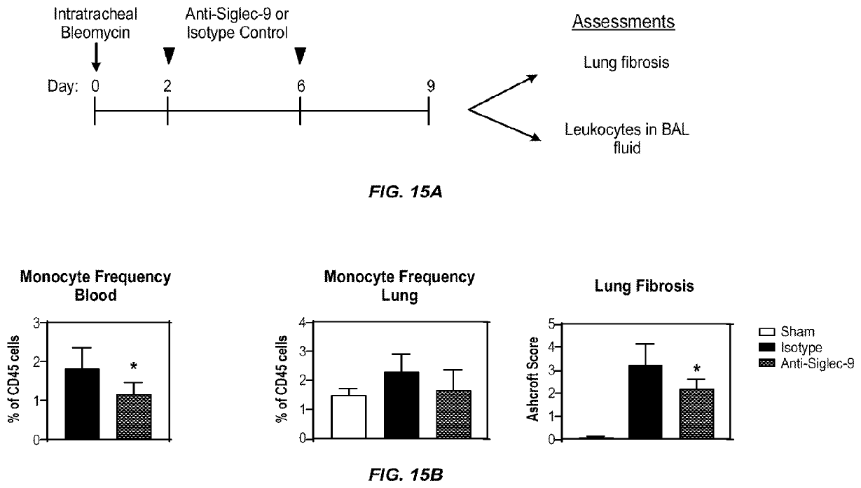

Antibody 9A12 was evaluated in an acute bleomycin-induced lung fibrosis model (derived form Yanabaetal., J. Immunol. 185:2502-2015, 2010). Oropharyngeal bleomycin was administered to mice. At time points two and six days following bleomycin administration, antibody (anti-Siglec 9 agonist antibody (9A12) or an isotype matched control was administered. The results demonstrate that dosing with an agonistic antibody reduced the frequency of monocytes in peripheral blood and lungs and led to reduced tissue damage in the lungs as quantified by Ashcroft scores (FIG. 15).

### Example 9. Mapping Siglec-9 Residues that are Important for Antibody Binding

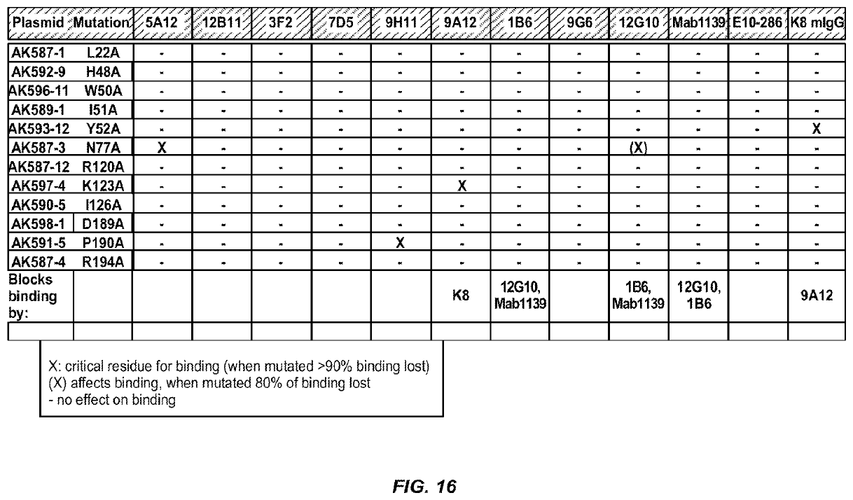

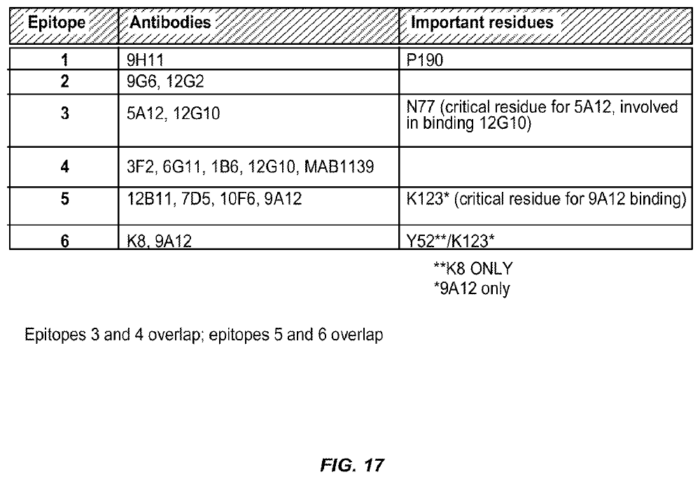

An alanine mutation analysis was used to identify residues important for binding for various monoclonal antibodies. The results are shown in FIGS. 16 and 17. Residue K123 of Siglec-9 was identified as a residue important for binding of antibody 9A12, but substitutions of L22, H48, W50, 151, Y52, N77, R120, 1126, D189, P190 or R194 with alanine exhibited no effects on binding. Residue N77 of Siglec-9 was identified as a residue important for binding of antibody 5A12, but substitutions of L22, H48, W50, 151, Y52, R120, K123, 1126, D189, P190 and R194 with alanine exhibited no effects on binding. Substitution of N77 of Siglec-9 with alanine also affected binding of 12G10 to Siglec-9, whereas substitution of L22, H48, W50, 151, Y52, R120, K123, 1126, D189,P190 or R194with alanine exhibited no effects on binding. Residue P190 of Siglec-9 was identified as a residue important for binding of antibody 5A12, but substitutions of L22, H48, W50, 151, Y52, N77, R120, K123, 1126, D189, and R194 with alanine exhibited no effects on binding.

### Example 10. Engineered Sequences

Mouse (m9A12) CDR sequences were identified as defined by IMGT (www imgtorg) and are underlined in the following sequence. A reference Fab AK0527-4 was constructed by fusing the intact mouse V-regions from m9A12 with mouse constant regions. A chimeric Fab AK0659-1 was constructed by fusing the intact murine V-regions from m9A12 with human constant regions. The CDR sequence are underlined below.

9A12 VH
(SEQ ID NO: 1)
QVQLQQSGAELVRPGASVTLSCKASDYTFTDYEMHWVKQTPVHGLEWIGA
IDPEAGGTDYNQKFKGKAILTADKSSSTAYMELRSLTSEDSAVYYCTRVW
LHWGQGTTLTVSS
9A12 Vk
(SEQ ID NO: 2)
NIVMTQSPSSLAVSAGEKVTMNCKSSQSVLYSSNQKNYLAWYQQKPGQSP
KMLIYWASTRESGVPDRFTGSGSGTDFTLTISSVQSEDLAVYYCHQYLSS
NTFGGGTKLEIK

The AK0527-4 and AK0659-1 Fabs were expressed in Expi293 cells, purified from cell media and tested by ForteBio kinetic analysis. The monovalent binding kinetics for the reference (mFab) and chimeric Fab (chFab) are summarized in Table 2

TABLE 2
ForteBio kinetic analysis of reference (mouse; mFab) and
chimeric (chFab) binding to SIGLEC-9-ECD-Fc antigen.

| Fab | Plasmid | KD (M) | Ka (1/Ms) | Kd (1/s) |
|---|---|---|---|---|
| 9A12 mFab | AK0527-4 | 6.66E−10 | 2.86E+05 | 1.90E−04 |
| 9A12 chFab | AK0659-1 | 5.68E−10 | 2.52E+05 | 1.43E−04 |

Gene sequences encoding the V-regions of 9A12(m9A12) that bind to human SIGLEC-9-ECD-Fc recombinant fusion protein (hereafter referred to as the antigen) were sub-cloned from PCR-amplified reactions. The m9A12 V-regions were humanized according to established procedures (Almagro & Fransson, J. Front Biosci 13:1619-1633, 2008; Lo, B. K. Methods Mol Biol 248, 135-159, 2004). The m9A12 HCDR and LCDR sequences were incorporated into the human germ-line sequences Vh 1-02 and VkIV B3 for the heavy and light chains, respectively. Additionally, selected CDR and framework mutations were introduced in order to improve affinity and to increase the percent identity to human germline. The FR4 sequence for the heavy chain is identical to human JH1 and the FR4 region for the light chain is identical to human Jk2.

V-regions containing framework and CDR mutations were generated by PCR mutagenesis. Each V-region was cloned into a vector containing the human IgG4 CH1 constant region for heavy chains or the human kappa constant region for light chains. For each test Fab, a heavy chain expression plasmid and a light chain expression plasmid were mixed and transfected into Expi293 cells. The secreted Fabs were purified from expression media and tested for antigen binding by ForteBio kinetic analysis. The monovalent binding kinetics for the CDR mutations tested are shown in Table 3.

TABLE 3
Antigen binding kinetics for the HCDR and LCDR mutations. The amino acid
sequences for the complete V-regions are shown in FIG. 2a and 2b. The monovalent
binding affinities were determined by ForteBio kinetic analysis. The residues shaded green
are identical to human germline and the residues shaded red differ from human germ-line
and are identical to the corresponding m9A12 sequence. NB = No detectable binding.

| Fab | Heavy Chain | Light Chain | Description | KD (M) | kon(1/Ms) | kdis(1/s) |
|---|---|---|---|---|---|---|
| Tf71 | AK641-2 | AK628-1 | HCDR1: GYTFTDYE | 5.33E−10 | 4.35E+05 | 2.32E−04 |
| Tf72 | AK642-1 | AK628-1 | HCDR1: DYTFTDYY | NB | NB | NB |
| Tf73 | AK643-3 | AK628-1 | HCDR1: DYTFTGYE | 5.75E−10 | 3.19E+05 | 1.84E−04 |
| Tf83 | AK645-12 | AK628-1 | HCDR2: INPEAGGT | 9.99E−09 | 2.98E+05 | 2.97E−03 |
| Tf84 | AK645-24 | AK628-1 | HCDR2: INPESGGT | 2.24E−09 | 1.32E+06 | 2.95E−03 |
| Tf85 | AK646-2 | AK628-1 | HCDR2: INPNSGGT | NB | NB | NB |
| Tf86 | AK646-4 | AK628-1 | HCDR2: INPNAGGT | NB | NB | NB |
| Tf87 | AK646-6 | AK628-1 | HCDR2: IDPNSGGT | 8.88E−09 | 6.28E+05 | 5.57E−03 |
| Tf88 | AK646-13 | AK628-1 | HCDR2: IDPNAGGT | 6.74E−09 | 2.72E+05 | 1.84E−03 |
| Tf74 | AK639-12 | AK644-3 | LCDR1: QSVLYSSNNKNY | 7.02E−10 | 3.32E+05 | 2.33E−04 |

V-regions that showed high affinity binding to SIGLEC-9-ECD antigen and high identity to human germline were chosen as lead candidates. Heavy and light chain V-region sequences that showed high affinity binding to SIGLEC-9-ECD antigen and high identity to human germline are are shown below for Fabs designated as Tf83 and Tf63”

Tf73 Vh
QVQLVQSGAEVKKPGASVKVSCKASDYTFTGYEMHWVRQAPGQGLEWMGA
IDPEAGGTDYAQKFQGRVTMTADTSISTAYMELSRLRSDDTAVYYCTRVW
LHWGQGTLVTVSS
Tf73 Vk
DIVMTQSPDSLAVSLGERVTMNCKSSQSVLYSSNQKNYLAWYQQKPGQPP
KMLIYWASTRESGVPDRFSGSGSGTDFTLTISSLQAEDVAVYYCHQYLSS
NTFGQGTKLEIK
Tf63 Vh
QVQLVQSGAEVKKPGASVKVSCKASDYTFTDYEMHWVRQAPGQGLEWMGA
IDPEAGGTDYAQKFQGRVTMTADTSISTAYMELSRLRSDDTAVYYCTRVW
LHWGQGTLVTVSS
Tf63 Vk
DIVMTQSPDSLAVSLGERVTMNCKSSQSVLYSSNQKNYLAWYQQKPGQPP
KMLIYWASTRESGVPDRFSGSGSGTDFTLTISSLQAEDVAVYYCHQYLSS
NTFGQGTKLEIK

As shown in Table 4, Tf73 exhibited a monovalent affinity for the antigen comparable to that of a reference Fab containing V-region sequences from m9A12, as determined by ForteBio kinetic analysis. The V-regions from the humanized Fab were re-formatted for expression as a human IgG4. The V-regions of a second candidate also exhibited a high monovalent binding affinity.

TABLE 4
ForteBio kinetic analysis of Fab binding to SIGLEC-9-ECD-Fc

| Fab | Vh Chain | Vk Chain | KD (M) | Ka (1/Ms) | Kd (1/s) |
|---|---|---|---|---|---|
| 9A12 mFab | | | 6.66E−10 | 2.86E+05 | 1.90E−04 |
| 9A12 chFab | AK608-1 | AK530-1 | 5.68E−10 | 2.52E+05 | 1.43E−04 |
| Tf63 Fab | AK639-12 | AK628-1 | 6.60E−10 | 2.42E+05 | 1.60E−04 |
| Tf73 Fab | AK643-3 | AK628-1 | 5.75E−10 | 3.19E+05 | 1.84E−04 |

The percentage sequence identity of humanized V-segments and V-regions of Tf73 and Tf63 sequences relative to human germ-line sequence are shown in Table 5. A

TABLE 5
Tf73 contains a heavy chain and a light chain with a high
degree of sequence identity to human germ-line immunoglobulin.
The lead candidate humanized V-segments and V-regions show
a greater than 90% sequence identity to the corresponding
closest human germ-line sequences outside the unique CDR3
regions on each of the heavy and light chains

| | | Total V- | % Identity | Total V- | % Identity |
|---|---|---|---|---|---|
| | Mis- | Segment | to Human | Region | to Human |
| Chain | matches | Residues | V-Segment | Residues | V-Region |
| Tf73 | 9 | 98 | 90.8 | 110 | 91.8 |
| Heavy | | | | | |
| Chain | | | | | |
| Tf63 | 10 | 98 | 89.8 | 110 | 90.9 |
| Heavy | | | | | |
| Chain | | | | | |
| Tf73 & | 8 | 101 | 92.1 | 112 | 92.8 |
| Tf63 Light | | | | | |
| Chain | | | | | |

Tf73 humanized V-segments and V-regions show a greater than 90% sequence identity to the corresponding closest human germ-line sequences outside the unique CDR3 regions on each of the heavy and light chains. TF63 also exhibited a high percent identity.

### Methods

K_{D} measurements

Antibody binding analysis was carried out by bio-layer interferometry (ForteBio). The assay was conducted at 25° C. in 1× ForteBio Kinetics buffer (ForteBiol8-132) in ultrapure water. Antibodies were captured on anti-mouse kinetic sensors at 0.5 ug/mL; Siglec-9-ECD was used as analyte and diluted in assay buffer from 50 nM to 1.56 nM with 2x dilutions. Two-minute associations were conducted, followed by 10-minute dissociations. Results were determined relative to a control empty reference AHC sensor, and analyzed using ForteBio analysis software with 1:1 global fit parameters.

### Antibody Competition Using Fortebio

Siglec-9-ECD-huFc was captured on anti-human IgG kinetic sensors at 0.5 ug/ml under saturating conditions (15 min at 1 μg/ml), after which the competing antibody was tested for binding. Each anti-Siglec-9 antibody was tested both ways (i.e. as saturating antibody and as competing antibody) against all other antibodies. When the antibody on the sensor competes with the antibody in solution, no additional binding to the antigen is observed. When a binding signal is observed, the two antibodies bind to the antigen in a non-competitive manner.

### Ligand Blocking Assay

Human breast cancer cell line T47D, which expresses high level of ligands for Siglec-9, was used to determine the blocking activity of anti-Siglec-9 antibodies in a cell-based assay. Two-fold serial dilutions of anti-Siglec-9 antibodies (40 nM to 40 pM) were combined with 10 nM Siglec-9-ECD-Fc in FACS buffer (PBS/2% BSA) and incubated on ice for 30 minutes. 2.5×10e4 T47D cells per well were added to round bottom 96-well tissue culture plates in FACS buffer, plates were centrifuged at 400g for 2 min, supernatant was removed and cells were re-suspended in 100 ul of the antibody-Siglec-9 complexes. After a 1 hour incubation on ice, cells were washed twice and incubated for 30 minutes with goat anti-human F(ab′)₂ fragment conjugated to AF647 (Jackson IR labs) at lug/ml in FACS buffer. After two more washes, cells were fixed in PBS/2% PFA and acquired on a Novocyte flow cytometer (ACEA biosciences). IC₅₀ values for blocking were determined based on plotting mean fluorescence in the APC channel and analysis using Prism Graphpad software.

### Ligand Displacement Assay

T47D cells were treated with 2ug/mL S9-ECD-Fc for 1 hour on ice. After the 1 hr preincubation of S9, a titration curve of anti Siglec-9 msIgG1s were added for 1 hr incubation at +37° C. Cells were then washed to remove unbound S9. Bound S9 were detected through the human Fc using Alexa488 conjugated secondary. Levels of remaining S9 were assessed by flow cytometry”

### Internalization Assay

Healthy donor peripheral blood mononuclear monocytes (PBMC) were used to determine internalization activity of anti-Siglec-9 antibodies. Previously cryopreserved PBMC were thawed and incubated for 90 minutes at 37° C. in complete medium (RPMI medium supplemented with 10% fetal bovine serum). Five-fold serial dilutions of anti-Siglec-9 antibodies (100 nM-0.01 pM) were prepared in complete medium. Cells (5×10e4 per well) and antibody dilutions were combined in 96-well tissue culture plates and incubated at 37° C. for 24 hours. Cells were re-suspended in huFc block (Becton Dickinson) in FACS buffer and stained with an antibody cocktail containing CD14-FITC and anti-Siglec-9 antibody 9H11-AF647 for 1 hour on ice. After two washes, cells were fixed in PBS/2% PFA and acquired on a Novocyte flow cytometer. IC₅₀ values for internalization were determined based on plotting mean fluorescence in the APC channel on monocytes (gated as CD3+) and analysis using Prism Graphpad software.

### Primary Tumor Analysis

Single cells from primary human tumor specimens were prepared using the Mylteni GentleMACS instrument according to the manufacturer's instructions. Cells were re-suspended in huFc block (Becton Dickinson) in FACS buffer and staining was performed using cocktails containing conjugated antibodies against immune cell surface markers, including CD3, CD8, CD16, CD45 and 7-AAD as a viability marker. CD8+ T cells were identified and gated as 7-AAD-CD45+CD3+CD8+. Anti-Siglec-9-PE (MAB1139 (R&D Systems) was used to detect Siglec-9 levels. Cells were fixed in PBS/2% PFA and acquired on a Novocyte cytometer. Gating and analysis was performed using Flowjo software (Tristar).

Ligand levels on tumor cells were detected using Siglec-9-ECD-Fc as described under Ligand blocking assay above. As a specificity control, cells were treated with sialidase/neuraminidase (Roche) at 0. 1U/mL to remove sialic acids from the cell surface.

Monocyte Derived Dendritic Cell (moDC) Differentiation Assay

Monocytes were purified from whole blood using a negative selection kit (Mylteni) according to the manufacturer's protocol and differentiated into dendritic cells using GM-CSF and IL-4 (both at 50 ng/mL) for 6 days and matured using LPS (100 ng/mL) or TNFa (50 ng/mL) for 2 days. Fully differentiated DCs were counted, stained for surface markers by flow cytometry and used in mixed lymphocyte reactions or antigen presentation assays.

### Mixed Lymphocyte Reaction

Monocyte derived dendritic cells were cultured with purified heterologous T cells at a 1:1 ratio for 5 days. T cell proliferation was assessed by cell counts on a flow cytometer.

### Autologous Antigen Presentation Assay

Matured moDCs from CMV positive donors were cocultured with autologous T cells, CEF peptide pool (JPT peptides) and IL-15 (50 ng/mL) for 3 days, then IL-2 (50 ng/mL) was added for 3 days. T cells were analyzed for CD107a and intracellular cytokines IFNγ, IL-2 and TNFα by flow cytometry (antibodies from BD using manufacturer's supplied protocol).

### Point Mutation Analysis to Define Siglec-9 Epitope Residues

Single amino acid mutations were generated in Siglec-9 expression plasmid and constructs were transfected into Expi293 cells according to the manufacturer's protocol (ThermoFisher). The next day the cells were stained with primary anti-S9 antibody at 2ug/mL for 30 minutes on ice in FACS buffer (PBS±2% BSA). Cells were washed with FACS buffer and binding was detected using an AF647 conjugated donkey anti-mouse antibody (Jackson Labs 715-606-151) and subsequent acquisition on a Novocyte flow cytometer.

### Macrophage Differentiation

Monocytes were purified from whole blood using a negative selection kit (Mylteni) according to the manufacturer's protocol and differentiated into macrophages using GM-CSF (50 ng/mL) for 6 days, followed by LPS (100 ng/mL)+IFNT(50 ng/mL) for 2 days (M1 phenotype), or M-CSF (50 ng/mL) for 6 days followed by either IL-4+IL-13(50 ng/mL) for 2 days (M2 phenotype). Differentiation was performed in the presence of anti-Siglec-9 or isotype control antibodies. Surface

### CD16+ Monocytes Isolation and Activation

CD16 positive monocytes were isolated from human peripheral blood using a positive selection kit (Myltenyi) and stimulated with LPS (0.5 ng/mL) for 20 hours in the presence of isotype control or anti-Siglec-9 antibody. Supernatants were collected and cytokine levels were determined using Meso Scale Discovery (U-plex) or Biolegend (Legendplex) kits.

### Autologous Monocyte-T Cell Co-Culture

CD16+ monocytes and T cells were isolated from PBMC using kits (from Miltenyi and Stemcell technologies, respectively) and plated in a T:mono ratio of 5:1. Isotype or anti-Siglec-9 antibody were added to the monocytes and subsequently the T cells and a CD3 (0. 1 ng/mL) and CD28 (lug/mL) antibody mixture were added. Supernatants were collected after 3 days and levels of cytokines were measured using a U-plex kit from MSD

### Bleomycin-Induced Fibrosis Model

Fibrosis was induced in C57BL6 Siglec-9 transgenic mice by intratracheal administration of 0.08 U bleomycin on Day 0, followed by treatment of isotype control or anti-Siglec-9 antibody on Day 2 and 6. Mice were taken down on Day 9 and the lungs of one half of each group (n=8) were analyzed by flow cytometry, while the other half (n=8) were fixed in formalin for quantification of lung injury (Ashcroft score).

### Engineering Antibody Sequences

### Sub-Cloning of Murine V-Regions

The V-region sequences from murine monoclonal antibody m9A12 were cloned by RT-PCR and cloned into an Allakos Fab expression vector. The plasmid AK0527-4 contains the mouse 9A12 V-regions and the mouse IgG1 CH1 and mouse Ckappa constant regions. The Fab expressed from AK0527-4 was tested for SIGLEC-9-ECD-Fc antigen binding and is referred to as reference sequence m9A12Fab in this report.

The m9A12 heavy and light variable regions were also combined with human IgG4 CH1 and human Ckappa constant regions in order to prepare a chimeric 9A12 (ch9A12) Fab expression construct (AK0659-1).

### Fab Preparation and Purification

Fab fragments were expressed by secretion from Expi293 cells. After transfection, the Expi293 cells were grown in Expi293 medium for two days at 37° C. with shaking. Secreted Fabs were purified via a 6XH is(H6)-tag appended to the C-terminus of the heavy chain CH1 region. Purified Fabs were quantified by protein gel electrophoresis and comparison to Fab standards of known concentration.

### Antigen Preparation

The gene region (accession number NM_0 14441; withoutintrons) containing the extracellular domain (amino acids 1-343; ECD) of human SIGLEC-9 was synthesized (Atum) and cloned into a mammalian expression vector and translationally fused with the human IgG1 Fc region. The SIGLEC-9-ECD-Fc fusion construct (AK0139-1) was transfected into CHOKl SV and stable cell lines expressing and secreting the fusion protein were obtained. The secreted SIGLEC-9-ECD-Fc fusion protein was purified from expression media by Protein A affinity capture.

### ForteBio Octet Red Kinetic Analysis

Anti-human Fc sensors were coated with SIGLEC-9-ECD-Fc at a concentration of 20 μg/ml in 1XHBS+1% BSA buffer (GE Lifesciences). After a brief wash with 1XHBS+1% BSA buffer, m9A12, ch9A12 or the humanized Fabs were applied to the antigen-coated sensors. The monovalent binding association and dissociation rates were calculated by the ForteBio Octet Red analysis software.

### Construction of IgGs

Chimeric 9A12 IgG4 was constructed by joining the human IgG4 (S228P; 3) and human kappa constant regions to the variable regions from the original mouse 9A12 antibody. The heavy chain variable region was cloned into a vector (AK0115-8) expressing the full-length IgG4 heavy chain expressed from a CMV promoter; the plasmid contains the AmpR gene for plasmid production in E. coli. Similarly, the light chain variable region was cloned into a vector (AKO111-7) containing the human kappa light chain constant region, expressed from a CMV promoter. The AKO111-7 plasmid contains the glutamine synthetase gene for selection in mammalian cells and the AmpR gene for selection in E. coli. The 9A12 heavy and light chains were then combined to result in the final IgG expression vector, named AK0533-2. Additional IgG expression constructs were prepared by a similar strategy for the humanized variants.

### Anti-Siglec-7 Binding Moiety

### Example 11. Binding Analysis of Anti-Siglec-7 Antibody

A panel of antibodies was evaluated for binding to Siglec-7 as described in U.S. Patent Application Publication No. 2019/0194323. The results identified anti-Siglec-7 antibody 16H11 having improved K_{D} values compared to previously described and/or commercially available QA79, S7.7, and Z176 anti-Siglec-7 antibodies (see, e.g., FIG. 7 of U.S. Patent Application Publication No. 2019/0194323).

Recombinant Siglec-7-Fc was added to A375 cells in the presence of anti-Siglec-7 antibodies at increasing concentrations and binding of the complex was detected on the cell surface as described in U.S. Patent Application Publication No. 2019/0194323. The results showed that antibody 16H11 blocked the interaction of Siglec-7 with ligands present on the surface of A375 melanoma human cells and demonstrated antibodies that have improved ligand blocking activity relative to previously described and/or commercially available QA79, S7.7, and Z176 anti-Siglec-7 antibodies (see, e.g., FIG. 8 of U.S. Patent Application Publication No. 2019/0194323.)

Internalization activity was also evaluated as described in U.S. Patent Application Publication No. 2019/0194323. Primary human peripheral blood mononuclear cells (PBMC) were incubated with increasing concentrations of anti-Siglec-7 antibodies for 24 hours and remaining Siglec-7 in the cell surface of NK cells was detected using a non-competing anti-Siglec-7 antibody. The results showed that 16H11 caused internalization of Siglec-7 on primary human NK cells and that 16H11 has improved internalization activity compared to commercially available and/or previously described QA79, S7.7, and Z176 anti-Siglec-7 antibodies (see, e.g., FIG. 9 of U.S. Patent Application Publication No. 2019/0194323).

### Example 6. Humanized Sequences

Illustrative humanized antibodies generated using monoclonal antibody 16H11 are described in U.S. Patent Application Publication No. 2019/0194323. Antibody binding results (measured in the form of a monovalent Fab) are shown in Table 1 of U.S. Patent Application Publication No. 2019/0194323. Antibodies listed in Table 1 of U.S. Patent Application Publication No. 2019/0194323 having a K_{D} of 500 pM or less are provided in Table 6 below. The sequence identifiers and sequences corresponding to the heavy and light chain “AK” number designations in Table 6 are provided in U.S. Patent Application No. 2019/0194323. Antibodies having the following heavy and light chain variable regions demonstrated K_{D}values (monovalent Fab) of about 75 nM or lower: VH438-4 and VL418-2; VH440-2 and VL418-2; VH441-2 and VL418-2; VH443-1 and VL418-2; VH444-2 and VL418-2, VH445-3 and VL418-2; VH449-4 and VL448-3; VH449-6 and VL418-2; VH387-11 and VL418-2; VH446-7 and VL418-2; VH 446-7 and VL448-3; VH463-2 and VL418-2; Vh463-2 and VL448-3; FH465-17 and VL418-2; VH484-6 and VL418-2; VH484-6 and VL448-3; and VH484-7 and VL448-3. The ligand blocking activity of the 16H11 anti-Siglec-7 antibody was also preserved

TABLE 6
K_{D} values of humanized antibodies derived from 16H11
measured as monovalent Fabs (from Table 1, U.S. Patent
Application Publication No. 2019/0194323):

| VH | VL | K_{D} (pM) |
|---|---|---|
| AK410-1 | AK418-2 | 250-500 |
| AK417-17 | AK418-2 | 250-500 |
| AK417-17 | AK421-3 | 250-500 |
| AK438-4 | AK418-2) | <75 |
| AK440-2 | AK418-2 | <100 |
| AK441-2 | AK418-2 | <75 |
| AK417-17 | AK435-7 | 250-500 |
| AK417-17 | AK439-5 | 250-500 |
| AK417-8 | AK439-5 | 250 |
| AK443-1 | AK418-2 | <75 |
| AK444-2 | AK418-2 | <75 |
| AK445-3 | AK418-2 | <75 |
| AK447-2 | AK418-2 | 75-150 |
| AK447-2 | AK448-3 | 75-150 |
| AK449-4 | AK418-2 | 75-150 |
| AK449-4 | AK448-3 | <100 |
| AK449-6 | AK418-2 | <100 |
| AK449-6 | AK448-3 | 50-100 |
| AK387-11 | AK418-2 | <75 |
| AK446-7 | AK418-2 | <75 |
| AK446-7 | AK448-3 | <75 |
| AK463-2 | AK418-2 | <75 |
| AK463-2 | AK448-3) | <75 |
| AK465-17 ( ) | AK418-2 ( | <75 |
| AK465-17 | AK419-2 | 150-250 |
| AK465-17 | AK448-3 | 100-150 |
| AK484-6 | AK418-2 | <100 |
| AK484-6 | AK448-3 | <75 |
| AK484-7 | AK448-3 | <75 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

Table of Illustrative variable region sequences

SEQ ID NO: 1 heavy chain variable region sequence of mouse anti-Siglec-9 antibody
(mouse antibody 9A12)
QVQLQQSGAELVRPGASVTLSCKASDYTFTDYEMHWVKQTPVHGLEWIGAIDPEAGGTDYNQKFKGKAI
LTADKSSSTAYMELRSLTSEDSAVYYCTRVWLHWGQGTTLTVSS
SEQ ID NO: 2 light chain variable region sequence of mouse anti-Siglec-9 antibody
(mouse antibody 9A12)
NIVMTQSPSSLAVSAGEKVTMNCKSSQSVLYSSNQKNYLAWYQQKPGQSPKMLIYWASTRESGVPDRFT
GSGSGTDFTLTISSVQSEDLAVYYCHQYLSSNTFGGGTKLEIK
SEQ ID NO: 6 Humanized V_{H} region 484-7 amino acid sequence—Siglec-7 binding
domain; CDRs as defined by Chothia are underlined
QVQLVQSGAEVKKPGSSVKVSCKASGYDFSNYWMNWVRQAPGQGLEWMGQIYPGD
GEIKYNQKFQGRVTITADESTSTAYMELSSLRSEDTAVYYCARDDYLRAMDYWGQG
TLVTVSS
SEQ ID NO: 7 Humanized V_{H} region 463-2 amino acid sequence—Siglec-7 binding
domain; CDRs as defined by Chothia are underlined
QVQLVQSGAEVKKPGSSVKVSCKASGYDFSNFWMNWVRQAPGQGLEWMGQIYPGD
GEIKYNQKFQGRVTITADESTSTAYMELSSLRSEDTAVYYCARDDYLRAMDYWGQG
TLVTVSS
SEQ ID NO: 8 Humanized V_{H} region 465-17 amino acid sequence—Siglec-7 binding
domain; CDRs as defined by Chothia are underlined
QVQLVQSGAEVKKPGSSVKVSCKASGYDFSNFWMNWVRQAPGQGLEWMGQIYPGD
GEIKYAQKFQGRVTITADESTSTAYMELSSLRSEDTAVYYCARDDYLRAMDIWGQGT
MVTVSS
SEQ ID NO: 9 Humanized V_{L} region 448-3 amino acid sequence—Siglec-7 binding
domain; CDRs as defined by Chothis are underline
DIQMTQSPSSLSASVGDRVTITCRASQNIHNYLAWYQQKPGKAPKFLLYSAKRLESGV
PSRFSGSGSGTDYTLTISSLQPEDFATYYCQHFWSSPYTFGQGTKLEIK
SEQ ID NO: 10 Humanized V_{L} region 418-2 amino acid sequence—Siglec-7 binding
domain; CDRs as defined by Chothia are underlined
DIQMTQSPSSLSASVGDRVTITCRASGNIHNYLAWYQQKPGKAPKFLLYSAKRLESGV
PSRFSGSGSGTDYTLTISSLQPEDFATYYCQHFWSSPYTFGGGTKVEIK
SEQ ID NO: 17—humanized VH region sequence Tf73—Siglec-9 binding domain
QVQLVQSGAEVKKPGASVKVSCKASDYTFTGYEMHWVRQAPGQGLEWMGAIDPEA
GGTDYAQKFQGRVTMTADTSISTAYMELSRLRSDDTAVYYCTRVWLHWGQGTLVTV
SS
SEQ ID NO: 18—humanized VL region sequence for Tf73 and TF63—Siglec-9 binding
domain
DIVMTQSPDSLAVSLGERVTMNCKSSQSVLYSSNQKNYLAWYQQKPGQPPKMLIYW
ASTRESGVPDRFSGSGSGTDFTLTISSLQAEDVAVYYCHQYLSSNTFGQGTKLEIK
SEQ ID NO: 19 humanized VH region sequence Tf63-Siglec—9 binding domain
QVQLVQSGAEVKKPGASVKVSCKASDYTFTDYEMHWVRQAPGQGLEWMGAIDPEA
GGTDYAQKFQGRVTMTADTSISTAYMELSRLRSDDTAVYYCTRVWLHWGQGTLVTV
SS
SEQ ID NO: 22 heavy chain variable region sequence (anti-Siglec-7 mouse monoclonal
antibody 16H11); CDRs as defined by Kabat: underline; CDRs as defined by Chothia:
bold, italics
QVQLHQSGAELVKPGASVKISCKGSGYDFSNFWMNWVKQRPGKGLEWIGQIYPGDGEIKYNGKFKGKATLTA
DESSSTAYIHLSSLTSEDSAVYFCARDDYLRAMDYWGQGTSVTVSS
SEQ ID NO: 23 light chain variable region sequence (anti-Siglec-7 mouse monoclonal
antibody 16H11); CDRs as defined by both Kabat and Chothia are underlined
DIQMTQSPASLSASVGETVTITCRASGNIHNYLAWFQQKQGKSPHFLVYSAKALADGVPSRFSGSGSGT
QYSLKINSLQPEDFGTYYCQHFWSSPYTFGGGTKLEIK

1. A mutlispecific antibody comprising a Siglec-9 binding domain and a Siglec-7 binding domain, wherein:
(a) the Siglec-9 binding domain comprises:
a V_{H} CDR1 comprising DYTFT(G/D)YE,
a V_{H} CDR2 comprising IDPEAGGT;
a V_{H} CDR3 comprising TRVWLH;
a V_{L} CDR1 comprising QSVLYSSNQKNY;
a V_{L} CDR2 comprising WAS, and
a V_{L} CDR3 comprising HQYLSSNT; or
a variant of the Siglec-9 binding domain in which one or more of the CDRs comprises one or two amino acid substitutions; and
(b) the Siglec-7 binding domain comprises:
a V_{H} CDR1 comprising GYDFSNF, GYTFSNF, GGDFSNF, GYDFSSY, GYDFSSF, or GYDFSNY;
a V_{H} CDR2 comprising YPGDGE, YPIDGE, or YPGFGE;
a V_{H} CDR3 comprising DDYLRAMD(Y/V/I);
a V_{L} CDR1 comprising RASGNIHNYLA, RASGGIHNYLA, RASQNIHNYLA, or RASGNISNYLA;
a V_{L} CDR2 comprising SAKRLES, AASRLES, SASRLES, SAKRLAS, or SAKRLED; and
a V_{L} CDR3 comprising QHFWSSPYT; or
a variant of the Siglec-7 binding domain in which one or more of the CDRs comprises an amino acid substitution.

2. A multispecific antibody of claim 1, wherein the Siglec-9 binding domain comprises a V_{H} CDR1 comprising DYTFT_G_/D)YE, a V_{H} CDR2 comprising IDPEAGGT; a V_{H} CDR3 comprising TRVWLH; a V_{L} CDR1 comprising QSVLYSSNQKNY; a V_{L} CDR2 comprising WAS, and a V_{L} CDR3 comprising HQYLSSNT.

3. The multispecific antibody of claim 1, wherein the Siglec-9 binding domain comprises a V_{H} region having at least 90% identity to amino acid sequence SEQ ID NO:17 or 19 and a V_{L} region having at least 90% identity to amino acid sequence SEQ ID NO: 18.

4. The multispecific antibody of claim 2, wherein the Siglec-9 binding domain comprises a V_{H} region comprising SEQ ID NO:17 or 19; and a V_{L} comprising SEQ ID NO:18.

5-6. (canceled)

7. The multispecific antibody of claim 2, wherein the Siglec-7 binding domain comprises:
a V_{H} CDR1 comprising GYDFSNF or GYDFSNY;
a V_{H} CDR2 comprising YPGDGE;
a V_{H} CDR3 comprising DDYLRAMD(Y/V/I);
a V_{L} CDR1 comprising RASGNIHNYLA or RASQNIHNYLA;
a V_{L} CDR2 comprising SAKRLES or SAKRLED; and
a V_{L} CDR3 comprising QHFWSSPYT.

8. The multispecific antibody of claim 7, wherein the Siglec-7 binding domain comprises a V_{H} CDR1 comprising GYDFSNF or GYDFSNY, a V_{H} CDR2 comprising YPGDGE; a V_{H} CDR3 comprising DDYLRAMDY or DDYLRAMDI, a V_{L} CDR1 comprising RASGNIHNYLA or RASQNIHNYLA, a V_{L} CDR2 comprising SAKRLES, and a V_{L} CDR3 comprising QHFWSSPYT.

9. The multispecific antibody of claim 8, wherein the Siglec-7 binding domain comprises a V_{H} CDR1 comprising GYDFSNY, a V_{H} CDR2 comprising YPGDGE; a V_{H} CDR3 comprising DDYLRAMDY, a V_{L} CDR1 comprising RASQNIHNYLA, a V_{L} CDR2 comprising SAKRLES, and a V_{L} CDR3 comprising QHFWSSPYT.

10. The multispecific antibody of claim 8, wherein the Siglec-7 binding domain comprises a V_{H} region having at least 90% identity to SEQ ID NO:6, 7, or 8; and/or a V_{L} region having at least 90% identity to SEQ ID NO:9 or 10.

11. The multispecific antibody of claim 10, wherein the Siglec-7 binding domain comprises a V_{H} of SEQ ID NO:6, 7, or 8; and/or the V_{L} region comprises SEQ ID NO:9 or 10.

12-15. (canceled)

16. The multispecific antibody of claim 1, wherein the antibody is a multivalent Fab form; or is a bispecific antibody.

17. (canceled)

18. The multispecific antibody of claim 1, comprising two or more human IgG Fc regions.

19. The multispecific antibody of claim 18, wherein the two or more human IgG Fc region comprises a mutation, relative to a native human IgG1 isotype sequence, that reduces binding of the antibody to an activating Fc receptor (FcR) and/or reduces Siglec-9 cross-linking: or the human IgG Fc region comprises a mutation, relative to a native human IgG1 isotype sequence, that enhances binding of the antibody to an Fc receptor (FcR) and/or enhances Siglec-9 cross-linking.

20. (canceled)

21. A pharmaceutical composition comprising an antibody of claim 1.

22. A method of inhibiting cancer cell proliferation, the method comprising administering a therapeutically effective amount of a neutralizing mutltispecific antibody of claim 1 to a subject that has a tumor comprising cancer cells and/or immune cells that express Siglec-9 and Siglec-7 ligands.

23. A method of inhibiting an immune response in a subject in need thereof, the method comprising administering a therapeutically effective amount of an agonist multispecific antibody of claim 1 to the subject.

24. (canceled)

25. An anti-Siglec-9 antibody that binds to an epitope in which residue K123 of human Siglec-9 as determined with reference to SEQ ID NO:16 is an important residue for binding to Siglec-9, but substitution of any one of residues L22, H48, W50, I51, or Y52 with alanine (A) has no effect on binding, wherein the anti-Siglec-9 antibody comprises a V_{H} region comprising a CDR1 sequence DYTFT(G/D)YE (SEQ ID NO:21), a CDR2 sequence IDPEAGGT (SEQ ID NO:12) and a CDR3 sequence TRVWLH (SEQ ID NO:13); and a V_{L} region comprising a sequence QSVLYSSNQKNY (SEQ ID NO:14), a CDR2 sequence WAS, and a CDR3 sequence HQYLSSNT (SEQ ID NO:8), or a variant of the antibody comprising one or more CDRs in which one amino acid is substituted.

26. (canceled)

27. The anti-Siglec-9 antibody of claim 25, comprising a V_{H} region having at least 90% identity to amino acid sequence SEQ ID NO:17 or 19 and a V_{L} region having at least 90% identity to amino acid sequence SEQ ID NO:18.

28-35. (canceled)

36. A pharmaceutical composition comprising an antibody of claim 25.

37. A method of inhibiting cancer cell proliferation, the method comprising administering a therapeutically effective amount of a neutralizing antibody of claim 25 to a subject that has a tumor comprising cancer cells and/or immune cells that express Siglec-9 ligands.

38. A method of inhibiting an immune response in a subject in need thereof, the method comprising administering a therapeutically effective amount of an agonst antibody of claim 25 to the subject.

39. The method of claim 38, wherein the subject has an autoimmune disease.

40. A nucleic acid encoding a V_{H} region and/or a V_{L}region as set forth in claim 25.

41. (canceled)

42. A host cell comprising the nucleic acid of claim 40.

43. (canceled)
